(19) United States
(12) Patent Application Publication
(10) Pub. No.: US 2003/0235580 A1
(43) Pub. Date: Dec. 25, 2003

(54) Amniotic membrane mediated delivery of bioactive molecules

(76) Inventor: Fen Zhang, San Francisco, CA (US)

Correspondence Address: PERKINS COIE LLP, P.O. BOX 2168, MENLO PARK, CA, 94026, US

(21) Appl. No.: 10/603,385

(22) Filed: Jun. 24, 2003

Related U.S. Application Data
(60) Provisional application No. 60/391,550, filed on Jun. 24, 2002.

(51) Int. Cl. A61K 39/395

(52) U.S. Cl. 424/130.1

(57) ABSTRACT

The present invention provides reconstituted and recombinant tissue membranes and methods for pharmaceutical delivery of bioactive molecules. In particular, reconstituted and recombinant amniotic membranes are provided for sustained delivery of therapeutic molecules, proteins or metabolites, to a site of a host in need thereof. The reconstituted and recombinant amniotic membrane contains one or more recombinant expression vectors that are exogenous to the membrane and capable of expressing bioactive molecules. The reconstituted and recombinant tissue membranes and methods can be used for in situ delivery of therapeutic proteins to a host in the treatment of disorders such as chronic wounds and dermatologic or ocular surface diseases.

This application claims priority benefit of U.S. Provisional Application No. 60/391,550, filed Jun. 24, 2002, which is incorporated herein in its entirety.

### FIELD OF THE INVENTION

The present invention relates to methods and compositions for delivering molecules to the skin of a recipient for various purposes, including therapy.

### REFERENCES

1. Phillips, T., B. Stanton, and A. Provan, A study of the impact of leg ulcers on quality of life: financial, social, and psychologic implications. J Am Acad Dermatol, 1994.31: p. 49-53.

2. Wound Care Specialization: An Important Trend Now Recognized as Board Certified Specialty. 2002, American Academy of Wound Management Press Release.

3. The US. Wound Management Products Market. 1996, Frost & Sullivan: Mountain View.

4. Backgrounder: Chronic Wounds. 2000, Human Genome Sciences.

5. Falanga, V. and M. Sabolinski, A bilayered living skin construct (APLIGRAF) accelerates complete closure of hard-to-heal venous ulcers. Wound Repair Regen, 1999. 7(4): p. 201-7.

6. Wieman, T. J., Clinical efficacy of becaplermin (rhPDGF-BB) gel. Becaplermin Gel Studies Group. Am J Surg, 1998. 176(2A Suppl): p. 74S-79S.

7. Cooper, D. M., et al., Determination of endogenous cytokines in chronic wounds. Ann Surg, 1994. 219(6): p. 688-91; discussion 691-2.

8. Kunimoto, B. T., Growth factors in wound healing: the next great innovation? Ostomy Wound Manage, 1999. 45(8): p. 56-64; quiz 65-6.

9. Smiell, J. M., Clinical safety of becaplermin (rhPDGF-BB) gel. Becaplermin Studies Group. Am J Surg, 1998. 176(2A Suppl): p. 68S-73 S.

10. Ravanti, L. and V. M. Kahari, Matrix metalloproteinases in wound repair (review). Int J Mol Med, 2000. 6(4): p. 391-407.

11. Lauer, G., et al., Expression and proteolysis of vascular endothelial growth factor is increased in chronic wounds. J Invest Dermatol, 2000. 115(1): p. 12-8.

12. Staiano-Coico, L., et al., Wound fluids: a reflection of the state of healing. Ostomy Wound Manage, 2000. 46(1A Suppl): p. 85S-93S; quiz 94S-95S.

13. Trengove, N. J., et al., Analysis of the acute and chronic wound environments: the role of proteases and their inhibitors. Wound Repair Regen, 1999. 7(6): p. 442-52.

14. Kim, J. C. and S. C. Tseng, Transplantation of preserved human amniotic membrane for surface reconstruction in severely damaged rabbit corneas. Cornea, 1995. 14(5): p. 473-84.

15. Akle, C. A., et al., Immunogenicity of human amniotic epithelial cells after transplantation into volunteers. Lancet, 1981. 2(8254): p. 1003-5.

16. Koizumi, N., et al., Cultivation of corneal epithelial cells on intact and denuded human amniotic membrane. Invest Ophthalmol Vis Sci, 2000. 41(9): p. 2506-13.

17. Ahn, S. T. and T. A. Mustoe, Effects of ischemia on ulcer wound healing: a new model in the rabbit ear. Ann Plast Surg, 1990. 24(1): p. 17-23.

18. Hao, Y., et al., Identification of antiangiogenic and antiinflammatory proteins in human amniotic membrane. Cornea, 2000. 19(3): p. 348-52.

19. Bodnar, A. G., et al., Extension of life-span by introduction of telomerase into normal human cells [see comments]. Science, 1998. 279(5349): p. 349-52.

20. Jiang, X. R., et al., Telomerase expression in human somatic cells does not induce changes associated with a transformed phenotype. Nat Genet, 1999. 21(1): p. 111-4.

21. Morales, C. P., et al., Absence of cancer-associated changes in human fibroblasts immortalized with telomerase. Nat Genet, 1999. 21(1): p. 115-8.

22. Tiberghien, P., “Suicide” gene for the control of graft-versus-host disease. Curr Opin Hematol, 1998. 5(6): p. 478-82.

23. Chalfie, M., et al., Green fluorescent protein as a marker for gene expression. Science, 1994. 263(5148): p. 802-5.

24. Berger, J., et al., Secreted placental alkaline phosphatase: a powerful new quantitative indicator of gene expression in eukaryotic cells. Gene, 1988. 66(1): p. 1-10.

25. Huter, O., et al., Lipoprotein lipase, LDL receptors and apo-lipoproteins in human fetal membranes at term. Placenta, 1997. 18(8): p. 707-15.

### BACKGROUND OF THE INVENTION

Chronic dermal wounds are a family of diseases that afflict about 2.5 million Americans each year [1]. The most prominent of these diseases include venous stasis ulcers, pressure ulcers and diabetic foot ulcers. A recent report by Frost and Sullivan estimates that the number of these wounds is increasing at a rate of 10 percent per year [2, 3]. The presence of underlying disease results in diminished ability for the body to heal injured tissue. As a result, chronic wounds are common among the elderly and individuals suffering from other conditions. Chronic wounds often take months or even years (sometimes even decades) to heal and often recur [4]. Approximately 50% of chronic wound patients are not cured by any existing treatment [5, 6].

Mechanisms of Chronic Wound Healing

The dermal wound could be the results of various types of trauma, infection, and inflammatory immune reactions. Wound healing is a complex process involving multiple steps such as coagulation, inflammation, angiogenesis, fibroplasia, matrix synthesis and deposition, contraction, epithelialization, and remodeling. The growth factors, various cytokines and proteases play important roles in promoting or modulating wound healing processes.

Chronic wounds are caused by a group of the factors that impede normal wound healing process. Some of the most commonly encountered and clinically significant impediments to wound healing including wound hypoxia, infection, hyperactive inflammation. Conventionally, treatment of chronic wounds frequently involves surgical debridement to convert the chronic wound into an acute wound. The main events of normal healing process for acute wounds include proliferation, migration, and differentiation. The present invention provides novel bioactive materials and methods for correcting many of the impediments to wound healing with therapeutic proteins, thereby restoring and promoting the normal wound healing process.

In a hypoxic condition, fibroblasts cannot replicate and collagen production is severely limited. Furthermore, wound hypoxia also predisposes the wound to bacterial invasion that significantly impedes wound healing. In addition to the surgical intervention for restoring the circulation to the wound sites, the main process of restoring of circulation is through angiogenesis at the wound sites. The inventor believes that sustained presence of growth factors for promoting angiogenesis at the wound sites should restore the circulation and correct the hypoxic condition.

Wound infection has been shown to impair wound healing in both acute and chronic wounds. High levels of bacteria have been demonstrated to impair every process in the wound healing scheme. Bacteria produce proteases capable of degrading extracellular matrix (ECM) as well as growth factors. The ECM, especially fibronectin, is required for fibroblast migration, and is also necessary to maintain macrophage phagocytic activity. Vitronectin is believed to regulate the fibrinolytic balance present at the advancing margin of new endothelial cells during angiogenesis. In addition, high levels of bacteria increase the number of polymorphonuclear leukocytes in the wound, which results in further increases in the level of proteases. Levels of MMP are also high in chronic wounds and decrease endogenous growth factor levels and function. Therefore, wound bacteria need to be controlled to levels of 10 bacteria per gram of tissue or fewer, with no tissue levels of beta-hemolytic streptococci. The inventor believes that delivery of antimicrobial proteins to the wounds should suppress bacterial infection at the wound sites.

Inflammation is part of the normal physiological process of wound healing. However, hyperactive inflammation, either due to infection or other persistent irritant at the wound site, is responsible to the excessive proteolytic activity, which is could lead to delay in wound healing process. Therefore, the inventor believes that under the condition that the infection has been brought under control and the irritant has been removed, delivery of anti-inflammatory proteins should suppress hyperactive inflammation, thereby promoting the wound healing process. Furthermore, the inventor believes that delivery of protease inhibitors should suppress excessive proteolytic activity at the wound site, thereby promoting the wound healing process.

Growth Factors Delivered by the Recombinant Tissue Membrane

Growth factors play important role in regulating every steps of wound healing process. It has been proven repeatedly that growth factors can promote wound healing in vitro, and in some acute experimental wounds in animals. Research indicates that repeated trauma and infection decrease the level of growth factors at the wound sites [7]. Indeed, it has been reported that platelet-derived growth factors (PDGF), basic fibroblast growth factor (FGF-2), epidermal growth factor (EGF), and transforming growth factor-b (TGF-b) levels are markedly decreased in wound fluid from chronic pressure ulcers compared with acute wounds. It has been suggested that the low level of growth factors at chronic wound sites are due to a combination of excessive destruction at the disease site and limited supply of these factors. In venous stasis ulcers and diabetic ulcers, the growth factors are not readily accessible to the wound site due to sequestration. These results suggest a potential role for exogenous growth factors in treating chronic wounds in compensating reduced growth factors at chronic wound sites. With the notable exception of platelet-derived growth factor (PDGF-β) [8], however, minimal success has been achieved by many of the human clinical trials with growth factors conducted in the past.

In the clinic, delivery of therapeutics into the patients is accomplished by using various administration methods, such as oral, intravenous, transdermal administration, or by surgical implantation of stents encapsulating drugs. For delivery of therapeutic proteins, many formulations of proteins have been developed to stabilize the protein and maintain the bioactivity of the protein. In particular, in the treatment of wounds such as open wounds (e.g. burns, venous stasis ulcers, decubitus ulcers and diabetic ulcers), purified therapeutic proteins have been delivered to the wound sites by topical application. The therapeutic proteins are formulated in saline solution and in biodegradable gels such as hyaluronate gel, methylcellulose, and fibrin gel [9].

One of the current challenges for clinical treatment with therapeutic proteins is administration of the bioactive proteins in pharmaceutically efficacious doses. It has been well documented that excessive proteolytic activities from infection and inflammation are commonly observed at chronic wound sites [10-13]. Due to excessive proteolytic activity commonly observed at chronic wound sites, it is conceivable that infrequent topical application will not likely to overcome the destructive activity of proteases at wound sites. Only with a sustained delivery system, will it likely to overcome the excessive proteolytic activities commonly found at chronic wound sites. The inventor believes that sustained delivery of therapeutic proteins, such as growth factors, is believed to be essential for a successful treatment of diseases such as chronic wounds due to venous stasis ulcers, bedsore ulcers, and diabetic foot ulcers.

The inventor demonstrates in the present invention that recombinant tissue membranes, e.g. recombinant human amniotic membranes transduced with adenoviral vectors, can be used as an in situ “bioreactor” for the production of transgenic proteins at the dermal wound sites. The genetically modified amniotic membrane exhibits a unique property that it can survive and continuously produce transgene product under a nutrient-poor environment. Such unique feature of recombinant tissue membranes promises a long-term sustained protein delivery that cannot be achieved by traditional topical applications. In one application, the exogenous therapeutic proteins delivered by the recombinant or reconstituted amniotic membrane can be used to correct or alleviate the underlying mechanism of chronic wounds and promote wound healing process.

The use of reconstituted tissue membrane as a delivery vehicle for therapeutics is advantageous. The reconstituted tissue membrane is a versatile “live-factory” producing and delivering bioactive molecules to a site of the host where the membrane is applied to, yet not genetically modifying the host tissue. For example, in clinical treatment of wounds and other surface diseases, such a genetically engineered tissue membrane can be used for in situ delivery of a bioactive molecule such as a therapeutic protein to the wounds by expressing the protein in the cells growing in the membrane. Furthermore, the reconstituted tissue membrane minimized potential biohazards of infectious agents that may associate with freshly prepared tissue membrane.

In one embodiment of the present invention, the reconstituted, or recombinant tissue membrane contains one or more expression vectors that express growth factors in the cells of the membrane. Examples of growth factors include, but are not limited to native or modified PDGF, FGF-2, EGF, epiregulin, TGF-a (and other EGF family growth factors), keratinocyte growth factor, keratinocyte growth factor-2 (KGF-2), granulocyte-macrophage colony-stimulating factor (GM-CSF), TGF-b, insulin-like growth factor I (IGF-I), and human growth hormone (HGH).

Description of Related Art

Amniotic Membrane

The amniotic membrane is an avascular tissue that forms the innermost layer of the fetal membrane. It is composed of five layers: an epithelial monolayer, acellular basement membrane layer, compact layer, mesenchymal cell layer, and spongy layer (Bourne, G. L. (1960) The microscopic anatomy of the human amnion and chorion. Am J Obstet Gynecol, 79, 1070-73.). Amniotic membrane is a relatively safe product for human use. Amniotic membrane was used clinically as a wound dressing for burn patients and in other surgical procedures as early as 1910 (Bose, B. (1979) Burn wound dressing with human amniotic membrane. Ann R Coll Surg Engl, 61, 444-7.; Sawhney, C. P. (1989) Amniotic membrane as a biological dressing in the management of burns [see comments]. Burns, 15, 339-42, Thomson, P. D., Parks, D. H. (1981) Monitoring, banking, and clinical use of amnion as a burn wound dressing. Ann Plast Surg, 7, 354-6.), and was reported to promote epithelialization, reduce pain, and prevent infection.

Today, preserved amniotic membranes are still used extensively in the ophthalmic field [14]. Human amniotic membranes are nearly non-immunogenic after being transplanted into the human body [15]. However, live native amniotic membranes pose a biohazard with potential blood borne pathogen contamination. A reconstituted amniotic membrane can mitigate such concern by seeding prescreened pathogen-free amniotic epithelial cells onto a sterilized amniotic membrane substrate or other matrixes.

Expression Vectors for Bioactive Molecules

A wide variety of expression vectors are available for transferring genes encoding bioactive materials into the amniotic membrane cells. These expression vectors can be viral vectors, such as modified or recombinant retroviruses, adenoviruses, and adeno-associated viruses. The ability of certain viruses to enter cells via receptor-mediated endocytosis and to integrate into the host cell genome and express viral genes stably and efficiently have made them attractive candidates for the transfer of foreign genes into mammalian cells.

Alternatively, the expression vectors can be transfected to the cells via non-viral transfection routes, such as physical methods, such as electroporation, ultrasound and chemical methods, liposome-mediated, activated-dendrimer-mediated and calcium-phosphate transfection.

Retroviruses

Retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three gene, gag, pol, and env that code for capsidal proteins, polymerase enzyme, and envelope components, respectively. A sequence found upstream from the gag gene, termed psi, functions as a signal for packaging of the genome into virions. Two long terminal repeat (LTR) sequences are present at the 5′ and 3′ end of the viral genome. These contain strong promoter and enhancer sequence and are also required for integration in the host cell genome.

To construct a retroviral vector, a nucleic acid encoding a bioactive molecule, such as growth factor PDGF, is inserted into the viral genome in the place of certain viral sequences to produce a virus that is replication-defective. Virions containing the inserted gene are produced in a packaging cell line containing the gag, pol, and env genes but lacking the LTR and psi components. When a recombinant plasmid containing the inserted gene, together with the retroviral LTR and psi sequences is introduced into this cell line (by calcium phosphate precipitation for example), the psi sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells. According to the present invention, the retroviral vectors can be used to transduce cells of the tissue membrane, such as amniotic epithelial cells.

Lentiviral vector is a newly developed retroviral vector that can infect the postmitotic cells and integrate its gene into host genome.

Type V Adenovirus

In contrast to retroviruses, the infection of adenoviral DNA into host cells does not result in chromosomal integration because adenoviral DNA can replicate in an episomal manner without potential genotoxicity. Also, adenovirus is structurally stable, and no genome rearrangement has been detected after extensive amplification. Type V adenovirus can infect virtually all epithelial cells regardless of their cell cycle stage. So far, Type V adenoviral infection appears to be linked only to mild disease such as acute respiratory disease in human.

Type V adenovirus is particularly suitable for use a gene transfer vector because of its mid-sized genome, ease of manipulation, high titer, wide target cell-range, and high infectivity. Both ends of the viral genome contain 100-200 base pair inverted terminal repeats (ITL), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off. The products of the late genes, including the majority of the viral capsid proteins, are expressed only after significant processing of a single primary transcript issued by the major late promoter (MLP). The MLP is particularly efficient during the late phase of infection, and all the mRNAs issued form this promoter possess a 5′ tripartite leader (TL) sequence which makes them preferred mRNA for translation.

Generation and propagation of adenovirus vectors, which are replication deficient, are carried in a helper cell line. Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g. Vero cells or other monkey embryonic mesenchymal or epithelial cells.

### SUMMARY OF THE INVENTION

The present invention provides reconstituted tissue membranes, recombinant tissue membranes and methods for pharmaceutical delivery and surgical implantation of bioactive molecules into subjects in need thereof In particularly, the invention relates to reconstituted amniotic membrane and recombinant amniotic membrane methods for pharmaceutical delivery and surgical implantation of bioactive molecules to or near a site of the host in need of the bioactive molecules. The reconstituted tissue membrane or isolated recombinant contains one or more expression vectors that express bioactive molecules, such as growth factors, anti-microbial proteins, anti-inflammatory protein, protease inhibitors, or hair growth promoting factors. Examples of growth factors include, but are not limited to PDGF, FGF-2, EGF, KGF-2, GM-C SF, TGF-b, IGF-I, and HGH. Examples of anti-microbial proteins include, but are not limited to bactericidal/permeability-increasing protein, defensin, collectin, Granulysin, Protegrin-1, SMAP-29, lactoferrin, Calgranulin C. Example of anti-inflammatory proteins include, but are not limited to interleukin-1 receptor antagonist, interleukin-10 and soluble TNF receptor, and soluble CTLA4. Examples of protease inhibitors include, but are not limited TIMP-1, -2, -3, -4, PAI-1, PAI-2, and ecotin. Examples of hair growth promoting factors include, but are not limited to wnt and sonic hedgehog.

In one embodiment, reconstituted tissue membrane is constructed by combining an isolated and sterilized tissue membrane, preferably, the amniotic membrane, that is substantially free of epithelium and mesenchymal cells and recombinant epithelial cells, preferably, the amniotic epithelial cells, growing on the isolated tissue membrane, containing one or more recombinant expression vectors exogenous to the tissue membrane and capable of expressing bioactive molecules. The bioactive molecules are polypeptides and other metabolites. Under suitable conditions, the recombinant epithelial cell expresses the bioactive molecules constitutively or in a controlled manner. The recombinant epithelial cells may be derived from but not limited to amniotic epithelial cells, epidermal keratinocytes of skin, and limbal epithelial cells of ocular tissue.

In another embodiment, reconstituted tissue membrane is constructed by combining the recombinant epithelial cells and synthetic substrate or matrix, preferably, a collagen matrix reinforced by a synthetic biodegradable polymer scaffold or meshwork.

In yet another embodiment, an isolated recombinant tissue membrane is provided which comprises one or more recombinant expression vectors exogenous to the tissue membrane and capable of expressing bioactive molecules. Examples of the tissue membrane include, but are not limited to amnion, cerebral dura mater, fascia lata, and pericardium isolated from human or other animal sources. These recombinant tissues contain cells, such as epithelial cells of amniotic membrane, that are genetically modified by transfection or transduction of recombinant expression vectors that are exogenous to the tissue membrane. These expression vectors can be viral vectors, such as modified or recombinant retroviruses, adenoviruses, and adeno-associated viruses. Alternatively, the expression vector can be transfected to the cells via non-viral transfection routes, such as calcium phosphate precipitation, electroporation, direct micro-injection, liposome-mediated and dendrimer-mediated transfection.

In yet another embodiment, a method is provided for delivering therapeutics to a host. The method comprises: providing a reconstituted tissue membrane; and applying the reconstituted tissue membrane to a site of the host in need of the bioactive molecules. The reconstituted tissue membrane comprises: an isolated tissue membrane that is substantially free of epithelium or synthetic matrix; and recombinant epithelial cells growing on the isolated tissue membrane, containing expression vectors exogenous to the tissue membrane and capable of expressing bioactive molecules.

In yet another embodiment, a method is provided for delivering therapeutics to a host. The method comprises: providing a recombinant tissue membrane; and applying the recombinant tissue membrane to a site of the host in need of the bioactive molecules. The isolated recombinant tissue membrane contains one or more recombinant expression vectors exogenous to the tissue membrane and capable of expressing the bioactive molecules. Preferably, the recombinant tissue membrane is modified human amnion.

Another aspect of the present invention provides a method for constructing a reconstituted tissue membrane. The method comprises steps of preparing a plurality of cells, preparing at least one recombinant expression vector, preparing a coated supporting matrix to support growth of said cells and providing mechanical strength for the reconstituted tissue membrane, and transducing said cells by said recombinant expression vector exogenous to the tissue membrane and enabling said transduced cells to produce bioactive molecules. In one embodiment, the coated supporting matrix is formed by an isolated bio membrane that is substantially free of epithelium cells and mesenchymal cells. The isolated bio membrane is sterilized to ensure the isolated bio membrane substantially free of pathogen. The sterilization process can be a treatment with gamma ray or with nucleic acids cross-linking reagents followed by UV radation.

Yet another aspect of the present invention provides a method for delivering recombinant proteins to a host site. The method includes: providing a reconstituted tissue membrane, wherein the reconstituted tissue membrane comprises: a plurality of recombinant amniotic cells, at least one recombinant expression vector, a coated supporting matrix to support growth of said cells and providing mechanical strength for the reconstituted tissue membrane; positioning the recombinant amniotic cells adjacent to the host site; having the recombinant amniotic cells express one or more recombinant proteins; having one or more recombinant proteins diffuse from the cells to the host site, wherein the recombinant amniotic cells contain one or more expression vectors and are capable of expressing recombinant proteins.

### BRIEF DESCRIPTION OF THE DRAWINGS

FIG. 1. Freshly harvested amniotic membrane was transduced by a recombinant adenovirus encoding marker protein green fluorescent protein (GFP) in vitro. GFP expression in adenoviral-transduced amniotic mesenchymal cells at Day 4 of post-transduction in DMEM/5% FBS. The amniotic mesenchymal cells assume a fibroblast cell-like morphology. The amniotic epithelial cells are out of focus. This figure showed that mesenchymal cells can be transduced by adenoviral vector in situ.

FIG. 2. Freshly harvested amniotic membrane was transduced by a recombinant adenovirus encoding marker protein green fluorescent protein (GFP) in vitro. GFP expression in adenoviral-transduced amniotic epithelial cells at Day 4 of post-transduction in DMEM/5% FBS. The amniotic epithelial cells assume a polygonal morphology. This figure shown that the epithelial cell can be tranduced by adenoviral vecto in situ.

FIG. 3. GFP expression persisted in adenoviral-transduced amniotic epithelial cells after 21 days in nutrient-poor culture medium (PBS/1.5% BSA). No GFP expression persisted in amniotic mesenchymal cells after fours days in nutrient-poor medium.

FIG. 4. SDS-PAGE of ³⁵S-labeled amniotic membrane after six days in nutrient-poor culture medium. Lane 1: immunoprecipitation with control antibody against cadherin; lane 2: whole cell lysate without immunoprecipitation, GFP band (arrow head); lane 3: immunoprecipitation with antibody against GFP (arrow). This figure demonstrated that new proteins are being continuously produced in nutrient-poor culture medium. In addition, it demonstrated that adenoviral induced GFP is the most prominent protein being produced in nutrient-poor culture medium.

FIG. 5. Rabbit wound on ischemic ear 16 days after surgery. Left wound with control amniotic membrane. Right wound with MDCK-PDGF-β seeded amniotic membrane. This figure demonstrated that a sustained delivery of growth factor PDGF-bb helps the wound healing in ischemic rabbit ear wound.

FIG. 6. GFP signal from adenoviral-transduced amniotic membrane on rat corneal surface 24 hours after transplantation. Curvature of corneal surface causes most of amniotic cells out of focus. This figure demonstrated that it is feasibility to delivery molecule on ocular surface.

FIG. 7. GFP expression in adenoviral-transduced amniotic membrane 72 hours after application at rat dermal wound site. The rat was euthanized 5 minutes before this photograph was taken in order to minimize blurring effect of the pulsation of life animal. There is no reduction of GFP expression. This figure demonstrated that an adenoviral-transduced amniotic membrane could survive and function in the wound bed over several days.

FIG. 8. Retroviral transduced human amniotic epithelial cells on collagen matrix formed membrane. This figure demonstrated that human amniotic epithelial cells can be effectively transduced by retroviral vector.

### DETAILED DESCRIPTION OF THE INVENTION

Delivery Systems of the Invention

The present invention provides systems for the delivery of a molecule to a patient. Systems of the invention are also referred to herein as recombinant tissue membrane(s). In certain embodiments, the systems are capable of delivering a molecule to a human patient, an adult human patient, a pediatric human patient, a male human patient, a female human patient, an animal, livestock, a cow, a horse, a pig, a rabbit, a rat, a vertebrate, a monkey, a dog. In certain other embodiments, the systems are capable of delivering a molecule to a patient, for example, for the purpose of therapy, treatment, cosmetics, prophylaxis, diagnosis, or any other purpose. Thus, the term patient is used herein to denote the recipient of the molecule to be delivered, regardless of the purpose of such delivery. In certain embodiments, systems of the invention include cells capable of delivering a molecule to a patient. In certain other embodiments, cells used in the invention deliver a molecule to a patient and the cells, for example, produced that molecule, contained that molecule, expressed that molecule, or expressed a polynucleotide encoding that molecule at some time prior to delivery of the molecule to the patient. In certain embodiments, the cells used in the the systems of the invention were transfected at some time so they can express a molecule for delivery to a patient.

Cells Useful in the Systems of the Invention

The systems of the invention include cells capable of delivering a molecule to a patient. Cells useful in the delivery systems of the present invention include, for example, cells found in the amniotic membrane, epithelial cells, epithelial cells of the amniotic membrane. In addition, it could include any modified or unmodified cell that is non-immunogenic and capable of survive and function in nutrient-poor environment.

Methods to Induce Delivery of a Molecule by the Cells Used in the Invention

Cells used in the systems of the invention are capable of delivering a molecule to a patient. Various means are contemplated to facilitate delivery of a molecule by the cells used in the systems of the invention. In certain embodiments, the cells are transfected with a polynucleotide, for example, a polynucleotide encoding the molecule to be delivered, or a polynucleotide capable of upregulating the production or expression of the molecule to be delivered in the cells used in the invention. For example, cells used in the invention may be transfected with a polynucleotide that encodes the molecule to be delivered. Or, for example, the cells used in the invention may be transfected with a polynucleotide that includes sequences encoding the molecule to be delivered and that further includes one, several or all required regulatory sequences to facilitate expression of the molecule. The polynucleotide may also include additional regulatory sequences, for example, to further increase or otherwise modulate the delivery of the molecule. The polynucleotide and/or regulatory sequences are chosen to deliver the molecule to be delivered in the desired amount, at the desired time, with all desired modifications, with expression of the molecule in the right compartment, for example, intracellular expression, extracellular expression, secretion, release through cell lysis, and so on. Thus, in these embodiments, an exogenous polynucleotide is introduced into the cells used in the invention and the polynucleotide encodes the entire, or parts of or fragments of, the molecule to be delivered to the patient.

Regulatory sequences useful in the systems of the invention include promoters, enhancers, introns, splice signals, transcription initiation sites, poly-adenylation signals, transcription stop signals, promoter motifs like, for example, a TATA box, sequences encoding signal sequences to achieve expression of the molecule to be delivered in the desired cellular compartment or routing of that molecule to the desired cellular compartment, for example, the endoplasmatic reticulum. Examples of such regulatory sequences include CMV promoter, SV40 promoter, retroviral LTR, internal ribosome entry site, SV40 poly-adenylation signal, bovine growth hormone polyadenylation signal.

In certain other embodiments, cells used in the invention are made to produce or express the molecule to be delivered to the patient using an endogenous polynucleotide, for example, by up-regulating the expression of an endogenous polynucleotide, or by inducing the production of the molecule to be delivered through any kind of endogenous activity, for example, an enzyme activity. In certain embodiments, a cellular activity is up-regulated in the cells used in the invention by exposing the cells to some stimulus, including, for example, radiation, an inducer, a stimulator, an ion, a peptide, a protein, a polynucleotide, a virus, a certain pH, a certain temperature, and so on.

Vectors Useful in the Systems of the Invention

Recombinant vectors can be used in the systems of the invention include but not limited to non-viral vectors, such as plasmid, cosmid; and viral vectors, such as retroviral vectors, including Moloney murine leukemia virus derived vectors and lentivirus derived vectors, adenoviral vector, and EBV derived vectors.

Promoters Useful in the Systems of the Invention

Examples of promoters useful in the invention are constitutive promters, for example, CMV promoter, SV40 promoters, retroviral LTR promoters, EF promoter; and inducible promoters, for example, tetracycline inducible promoter, or more specific inducible promoters like inflammation induced promoter, such as TNF-a promoter, and IL-1 promoter.

Other Components in the Systems of the Invention

The systems of the invention may include various other components in addition to cells and a means to facilitate the delivery of a molecule to a patient. For example, the systems may include a medium in which the cells used in the invention are maintained or kept. Examples of such a medium include PBS, DMEM, any type cell culture medium. In certain embodiments, the systems of the invention may include a buffer, for example, HEPES buffer, a citrate based buffer, PBS. In certain embodiments, the systems of the invention may include a dye, packaging, instructions on how to use the systems, DMSO, glycerol for cryopreservation of the systems of the invention.

Application of the Systems of the Invention

Systems of the invention can be used for a variety of applications, for example, for therapy, diagnosis, cosmetics, and any other application where it is desired to deliver a molecule to the skin of a patient or recipient.

The Wound and Diabetic Foot Ulcer Markets

The Chronic Wound Market

The chronic wound market is large and growing. A recent report by Frost and Sullivan indicates the wound market has grown to a projected $2.57 billion in 2002 for products directly related to the treatment of these wounds. The total cost of treating chronic wounds is estimated at $5 to $7 billion annually. The report estimates that the number of these wounds is increasing at a rate of 10 percent per year [3]. Chronic wounds are a family of diseases that afflict about 2.5 million Americans each year [4]. A chronic wound has been defined as any disruption of the anatomic or physiologic functions of tissue, complicated by one or more chronic illnesses, and lasting more than three months [3]. The presence of underlying disease results in diminished ability for the body to heal injured tissue. As a result, chronic wounds are common among the elderly and individuals suffering from other conditions. Chronic wounds often take months or even years (sometimes even decades) to heal and often recur [4].

There are three major types of chronic wounds:

| | | | Prevalence: |
|---|---|---|---|
| Type of | | | Industrialized |
| Wound | Description | Prevalence: U.S. | World |
| Venous | An ulceration that | 500,000-700,000 | 1.3 million |
| stasis | develops on the ankle | people | people |
| ulcer | or lower leg in patients | | |
| | with chronic vascular | | |
| | disease | | |
| Diabetic | A chronic wound of | 500,000 people | 1.2 million |
| foot | the foot that occurs in | | people |
| ulcer | patients with diabetes | | |
| Pressure | Any lesion caused by | 1.6 million | 4.1 million |
| ulcer | unrelieved pressure on | people | people |
| | tissues that are located | | |
| | over a bony | | |
| | prominence on the | | |
| | body (i.e., “bedsores”) | | |

Source: Human Genome Sciences. Backgrounder: Chronic Wounds. September 2000. http://www.hgsi.com/news/press/background_wounds.html. Accessed March 10, 2002.

Other Market Opportunities

Amniotic membrane-mediated protein delivery systems can be adapted to the treatments of other chronic wounds and other dermatologic and ocular surface diseases. Near-term markets where this technology would be particularly advantageous include venous stasis ulcers; pressure ulcers; skin surgery (600,000/year), promoting wound healing; burns (12,000/year), promoting wound healing, suppressing inflammation and hypertrophic scarring; epidermolysis Bullosa (100,000/year); dry eye, suppressing inflammation; photorefractive surgery, promoting wound healing, suppressing corneal haze; corneal ulcers, promoting wound healing, suppressing infection and inflammation; corneal neovascularization and inflammatory diseases.

In the longer-term, application of this technology can be extended to cosmetic surgery: postoperative wound dressing after laser based skin cosmetic (wrinkle removing) surgery; hair growth: provision of growth factors, such as wnt, sonic hedgehog, keratinocyte growth factors and cytokines, in proximity to hair follicles to promote hair growth; others: implanted protein delivery device for the treatment of diseases such as severed nerve, fracture bones, anemia, or neuron degenerative diseases.

(Methods of Treating Diseases and Disorders

Systems of the present invention can be applied to a patient or recipient in a variety of ways known to those of skill in the art. For example, where it is desired to deliver a molecule to the skin of a patient to achieve a particular reaction, one may apply the system of the invention topically to the skin of interest. In this example, where the system of the invention consists of cells expressing the molecule to be delivered, the cells are topically applied to the skin. Such application may include the cells and other aspects of the systems of the invention. Where the cells of the system of the invention are applied to skin topically, the cells may be applied in such a quantity as to cover the entire skin area of interest in its entirety as a single cell layer, or, for example, as a double cell layer, a triple cell layer, or any other higher number of cell layers, or any broken number of cell layers. In another aspect of the invention, the cells may be applied in lesser quantities, for example, in a layer that is evenly spread over the entire skin area of interest and that covers less than the entire skin area of interest, in other words, a layer with gaps between the cells. In this aspect of the invention, the cell layer may cover 90 percent of the skin area of interest, or 80 percent, 70 percent, 60 percent, 50 percent, 25 percent, 10 percent, or any other percentage. The desired quantity of cells to apply to the skin area of interest depends on a number of factors, including the desired result of the application, the time in which the result is desired, the amount of the molecule to be delivered that each cell of the systems of the invention delivers to the skin, the condition of the skin that the cells are applied to.

Once cells of the systems of the invention are applied to the skin, one may observe the progress towards achieving the desired effect. Such progress may be observed, for example, through observation or by measuring the level of any kind of marker that is associated with the present condition of the skin and that is undesired, or one can measure the level of any marker that is associated with the desired condition of the skin that the systems of the invention are intended to realize.

Markers such as the green fluorescent protein (GFP) and secreted alkaline phosphatase (SEAP) produced by the cells of the systems that will reflect the health of cells of the system. Markers such as TNF-a, IL-1 and other inflammatory cytokines in the wound exude can be assayed to reflected the inflammatory status of wound bed. Markers such as proteinases, such MMPs and their inhibitor (TIMPs) in the wound exude can be used to assess the proteolytic environment at the disease sites. Markers such as growth factors and cytokines concentration at the disease sites can be used to monitor the efficacy of the treatment with the system of the invention. These markers can be examined by ELISA assays and enzymatic assays on harvested wound exudation or washed solution from the disease site. The other markers will be clinical score the of the disease states, such as redness, wound size, microbial counts, hair counts. The expression of a molecule in the cells and their dosages can be assay by enzymatic assay, for example, SEAP and ELISA assay of the molecule. It is more convenient and more sensitive to measure the expression level of SEAP with chemiluminescent assay to indirectly measure the production level of desired molecule with a pre-determined conversion chart established in a standard condition.

One of the major concerns of gene therapy is immuno-compatibility. The transduced cells should be able to secrete desired bioactive molecules, yet they should not be rejected by the host. The original cells before transduction should not be immuno-genic, nor should the cells be rendered immuno-genic after transduction. The same is true for the supporting matrix. The suitable materials for non-immuno-genic purpose are amniotic membrane and amniotic epithelial cells. Meanwhile, the dermal keratinocyte gene therapy system described in reference may have the risk to be rejected by the host.

Amniotic membrane, which is an avascular tissue, composed of five layers: an epithelial monolayer, acellular basement membrane layer, compact layer, mesenchymal cell layer, and spongy layer. It forms the innermost layer of the fetal membrane. It is used clinically as a wound dressing for burn patients and in other surgical procedures as early as 1910 (Bose, B. (1979) Burn wound dressing with human amniotic membrane. Ann R Coll Surg Engl, 61, 444-7; Sawhney, C. P. (1989). Burns, 15, 339-42, Thomson, P. D., Parks, D. H. (1981) Monitoring, banking, and clinical use of amnion as a burn wound dressing. Ann Plast Surg, 7, 354-6.). Amniotic membrane is reported to promote epithelialization, reduce pain, and prevent infection. Preserved amniotic membranes are still used extensively in the ophthalmic field today (14). Human amniotic membranes are nearly non-immunogenic after being transplanted into the human body (15). Amniotic membrane is safe for human use.

The genetically modified, reconstituted tissue membrane from amniotic membrane exhibits several unique properties. First, the reconstituted tissue membrane is a safe product free of potential blood borne pathogen contamination. Therefore, There is no secondary infection risk to the host when reconstituted tissue membrane is applied. Second, the reconstituted tissue membrane can be designed in vitro to produce various kinds of bioactive molecules by transducing cultured epithelial cells with different combination of expression vectors to address individual need of the patient. Third, the reconstituted tissue membrane components, the cells and the supporting matrix, are from amniotic membrane origin, which is non immunogenic. They remain non immunogenic after transduction and preparation process. There is no risk of host rejection. Fourth, the reconstituted tissue membrane delivers bioactive molecules in an avascular condition. The supporting matrix is coated, and the membrane is treated to be suitable sustain the epithelial cells in poor nutrient condition. There is no need for nutrient from blood supply. It survives and delivers bioactive molecules continuously in a poor nutrient environment, which is especially important to treat chronic wound. Fifth, the reconstituted tissue membrane can be prefabricated and adopted for mass production.

In one embodiment, reconstituted tissue membrane is constructed by preparing an isolated and sterilized bio membrane. The process starts with an amniotic membrane which includes five layers: epithelial cell monolayer, basement membrane, compact layer, mesenchymal cell layer, and sponge layer. The freshly isolated amniotic membranes are sterilized by lethal dose of gamma irradiation. Alternatively, the freshly isolated amniotic membranes are sterilized by nucleic acid cross-linking reagents, preferably, Cerus Corporation's Helinx® compounds followed by UV radiation. The membrane is now substantially free of pathogen. The treated amniotic membranes are stripped off of the epithelial cells and mesenchymal cells by incubating with 8 mM EDTA/EGTA in PBS for two hour at 37° C., followed by mechanical scraping with cell scrapers [16]. Such a sterilized amniotic membrane can then serve as the supporting matrix for cultured amniotic epithelial cells. The next step is to coat the isolated bio membrane with matrix protein laminin and fibronectin. This process produces a coated supporting matrix.

The resulting amniotic membranes are washed in culture medium and then placed on a hydrophobic surface with basement membrane facing up.) Cultured epithelial cells or its derived cell lines are then transfected or transduced with expression vectors exogenous to the epithelial cells and the membrane, resuspended and seeded onto the denuded amniotic membrane in a small volume. The epithelial cells are confined to a small area over the amniotic membrane by the hydrophobic surface to achieve a high seeding density. After cell attachment, the reconstituted amniotic membranes are transferred to a new dish and cultured until the epithelial cells reach confluency. The culture media is supplemented with extra lipids, which may prolong the survival of amniotic epithelial cells in a nutrient-poor environment.

Examples of the bio membrane include, but are not limited to amnion, cerebral dura mater, fascia lata, and pericardium isolated from human or other animal sources. Cultured recombinant epithelial cells, preferably the amniotic epithelial cells, transduced by at least one exogenous recombinant expression vector, are transferred and seeded onto the prepared bio membrane. These expression vectors can be viral vectors, such as modified or recombinant retroviruses, adenoviruses, adeno-associated viruses, lentivirus, adeno-associated viral vector, and plasmid. Alternatively, the recombinant expression vector can be a physically or chemically transduced vector. For example, the expression vector can be transfected to the cells via non-viral, physical and chemical transfection routes, such as calcium phosphate precipitation, electroporation, direct micro-injection, liposome-mediated and dendrimer-mediated transfection.

The reconstituted tissue membrane containing one or more recombinant expression vectors exogenous to the bio membrane is capable of expressing bioactive molecules and is ready for use on patient. Under suitable conditions, the recombinant epithelial cell expresses the bioactive molecules constitutively or in a controlled manner. The recombinant epithelial cells may be derived from but not limited to mammalian amniotic epithelial cells, epidermal keratinocytes of the skin, and limbal epithelial cells of ocular tissue such as corneal epithelial cells and conjunctival epithelial cells.

The reconstituted tissue membrane contains one or more expression vectors that express bioactive molecules, such as growth factors, anti-microbial proteins, anti-inflammatory protein, anti-neovascularization proteins, protease inhibitors, or hair growth promoting factors. The bioactive molecules are polypeptides and other metabolites. Examples of growth factors include, but are not limited to native or modified PDGF, PDGF-beta, FGF-2, EGF, VEGF, NGF, keratinocyte growth factor-2 (KGF-2), granulocyte-macrophage colony-stimulating factor (GM-CSF), TGF-b, insulin-like growth factor I (IGF-I), and human growth hormone (HGH). Examples of anti-microbial proteins include, but are not limited to bactericidal/permeability-increasing protein, defensin, collectin, Granulysin, Protegrin-1, SMAP-29, lactoferrin, Calgranulin C. Examples of anti-inflammatory proteins include, but are not limited to interleukin-1 receptor antagonist, interleukin-10 and soluble TNF receptor, and soluble CTLA4. Examples of anti-inflammatory proteins include, but are not limited to endostatin, angiostatin, soluble VEGF receptor, and TIMP-1, TIMP-2, TIMP-3, TIMP-4. Examples of protease inhibitors include, but are not limited to TIMP-1, TIMP-2, TIMP-3, TIMP-4, PAI-1, PAI-2, and Ecotin. Examples of hair growth promoting factors include, but are not limited to wnt and sonic hedgehog.

A wide variety of expression vectors are available for transferring genes encoding bioactive materials into the epithelial cells. These expression vectors can be viral vectors, such as modified or recombinant retroviruses, adenoviruses, and adeno-associated viruses. The ability of certain viruses to enter cells via receptor-mediated endocytosis and to integrate into the host cell genome and express viral genes stably and efficiently have made them attractive candidates for the transfer of foreign genes into mammalian cells.

Alternatively, the expression vectors can be transfected to the cells via non-viral transfection routes, such as physical methods electroporation, ultrasound or chemical methods liposome-mediated, activated-dendrimer-mediated and calcium-phosphate transfection.

Retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. Retroviral vector only transduce dividing cells. And it will not subject the transduced cells to immune response. On the other hand, the adnoviral vector transfected epithelial cells described in the reference may be immunogenic, and may be rejected by the host. The retroviral genome contains three gene, gag, pol, and env that code for capsidal proteins, polymerase enzyme, and envelope components, respectively. A sequence found upstream from the gag gene, termed psi, functions as a signal for packaging of the genome into virions. Two long terminal repeat (LTR) sequences are present at the 5′ and 3′ end of the viral genome. These contain strong promoter and enhancer sequence and are also required for integration in the host cell genome.

To construct a retroviral vector, a nucleic acid encoding a bioactive molecule, such as growth factor PDGF, is inserted into the viral genome in the place of certain viral sequences to produce a virus that is replication-defective. Virions containing the inserted gene are produced in a packaging cell line containing the gag, pol, and env genes but lacking the LTR and psi components. When a recombinant plasmid containing the inserted gene, together with the retroviral LTR and psi sequences is introduced into this cell line (by calcium phosphate precipitation for example), the psi sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells. According to the present invention, the retroviral vectors can be used to transduce cells of the bio membrane, such as amniotic epithelial cells.

Lentiviral vector is a newly developed retroviral vector that can infect the postmitotic cells and integrate its gene into host genome.

In contrast to retroviruses, the infection of adenoviral DNA into host cells does not result in chromosomal integration because adenoviral DNA can replicate in an episomal manner without potential genotoxicity. Also, adenovirus is structurally stable, and no genome rearrangement has been detected after extensive amplification. Type V adenovirus can infect virtually all epithelial cells regardless of their cell cycle stage. So far, Type V adenoviral infection appears to be linked only to mild disease such as acute respiratory disease in human.

Type V adenovirus is particularly suitable for use as a gene transfer vector because of its mid-sized genome, ease of manipulation, high titer, wide target cell-range, and high infectivity. Both ends of the viral genome contain 100-200 base pair inverted terminal repeats (ITL), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off. The products of the late genes, including the majority of the viral capsid proteins, are expressed only after significant processing of a single primary transcript issued by the major late promoter (MLP). The MLP is particularly efficient during the late phase of infection, and all the mRNAs issued form this promoter possess a 5′ tripartite leader (TL) sequence which makes them preferred mRNA for translation.

Generation and propagation of adenovirus vectors, which are replication deficient, are carried in a helper cell line. Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g. Vero cells or other monkey embryonic mesenchymal or epithelial cells.

In another embodiment, reconstituted tissue membrane is constructed by combining the recombinant epithelial cells and synthetic substrate or matrix, preferably, a collagen matrix reinforced by a synthetic biodegradable polymer scaffold or meshwork. The process starts with a crosslinked collagen I matrix. A first surface of the collagen I matrix is coated with fibronectin and vitronectin to enhance the attachment of epithelial cells on this surface. A second surface, which is cell-free surface of the collagen I matrix is coated with hyaluronate to reduce the cell attachment on this surface. The process produces a coated supporting matrix. Cultured epithelial cells or its derived cell lines are then transfected or transduced with expression vectors exogenous to the epithelial cells, resuspended and seeded onto the coated supporting matrix in a small volume. The epithelial cells are confined to a small area over the coated supporting matrix by the hydrophobic surface to achieve a high seeding density. After cell attachment, the reconstituted membranes are transferred to a new dish and cultured until the epithelial cells reach confluency. The culture media is supplemented with extra lipids, which may prolong the survival of amniotic epithelial cells in a nutrient-poor environment.

In yet another embodiment, a method is provided for delivering therapeutics to a host. The reconstituted tissue membrane comprises a plurality of epithelial cells grown on a first surface of a coated supporting matrix. The membrane is supported by a breathable substrate attached to a second surface of the coated supporting matrix. The membrane is applied onto the wound with the epithelial cells facing to the wound. The epithelial cells transduced with exogenous expressing vectors secrete bioactive molecules diffusing in to the wound.

The following experiment verifies the efficacy and safety of reconstituted amniotic membrane in a rabbit ear ischemic model. The ischemic dermal wound is created by ligating the rostral and central arteries of rabbit ears following a published method (17). Four 6-mm diameter dermal wounds to the depth of bare cartilage are created by a surgical punch biopsy instrument. The amniotic membranes are carefully placed onto the wound sites with epithelial cells facing down toward the wound site. The dosage of growth factors is adjusted by the size of transplanted amniotic membrane cut with appropriate size trephines. Wound sites are protected by a transparent polyurethane film wound dressing (Tegaderm).

The study is conducted in both ischemic and nonischemic wound sites on rabbit ears. The viability of the amniotic membrane is monitored by continuous GFP signals under a fluorescence microscope. The wound exudates are harvested with a glass capillary every 24 hours for 8 days. In order to collect the wound exudates on a more consistent level, 50 microliters of PBS are applied to each wound site 5 minutes before the wound exudates collected with the glass capillary. A new Tegaderm film is reapplied after each wound exudate sample collection. The transgene products concentration in wound exudates are assayed by monitoring SEAP enzyme activities by chemiluminescent technique. The profile of SEAP activity over the experimental period is parallel of that of secretion growth factor.

Isolation of human amniotic epithelial (HAE) cells is accomplished by trypsin digestion (18). HAE cells are genetically modified to express two marker proteins, GFP and SEAP, through retroviral vectors or transfection of plasmid DNA. The cytosolic marker, GFP, is lost if HAE cell is damaged or sloughed off from the reconstituted amniotic membrane. The secreted marker, SEAP, is used to quantify the transgene production by the reconstituted amniotic membrane. The secreted SEAP in the reconstituted amniotic membrane culture medium can be quantified by chemiluminescent assay.

A newly developed immortalizing technique based on human telomerase reverse transcriptase (hTERT) can avoid such potential adverse effects associated with SV40 T antigen [19-21]. hTERT, which is normally expressed only in embryonic stem cells, germline cells, and cancer cells, maintains the length of the telomeres, the DNA repeats at the end of chromosomes. The constitutively expressed hTERT is able to maintain the length of telomeres in cell lines. The expression of hTERT does not alter the cell cycle pathways as SV40 T antigen does. Therefore, telomerase immortalized cells do not exhibit the transformed phenotype. The expression of hTERT, in primary cultured HAE cells or corneal and conjunctival epithelial cells through retroviral vectors is used to establish immortalized cell lines. The immortalized HAE cells are further modified to express two marker proteins, GFP and SEAP, through retroviral vectors.

A successful wound healing may accidentally entrap some HAE cells at the wound sites. To ensure that immortalized HAE cells are not become tumors in the host, the HAE is further genetically modified to express suicide protein, Herpes Simplex Virus thymidine kinase (HSV-tk). Administering prodrug ganciclovir, which further ensures the safety of reconstituted amniotic membrane in clinical application, can destroy entrapped HSV-tk positive cells at wound site. The growth factor PDGF-b is selected to be expressed by HAE cells as a therapeutic protein to promote healing of ischemic wounds.

By genetically modifying human amniotic membrane cells, the genetically modified amniotic membrane is converted into a “live factory” or a “bioreactor”, which can manufacture and deliver therapeutic proteins in situ at the wound site.

Furthermore, owing to the versatility and exchangeability of the isolated membrane, the transgene production and delivery can be sustained for a long duration by simply replacing a used amniotic membrane with freshly prepared amniotic membrane when necessary. Therefore, this approach may overcome the obstacles encountered in conventional gene therapy approaches, such as short duration of transgene expression, and host immune response to the vector or the transgene itself.

In addition, the amniotic cells can be transduced by several expression vectors encoding for different therapeutic proteins simultaneously. This could make a combinational therapy a relatively easy task. Compared with many previous attempts, the novel biomaterials of the present invention can provide a sufficient quantity of therapeutic proteins for a sufficient length of times to effectively promote healing for chronic wounds.

The preferred adenoviral vector encodes two transgene units, one encoding for a reporter protein, the secreted form of human placental alkaline phosphatase (SEAP), the other encoding for specific therapeutic protein. SEAP activity can be monitored through a highly sensitive chemiluminescent assay. The assay can detect as little as 10⁻¹³ g of SEAP protein, and remains linear over a 10,000 fold range of protein concentration. The amount therapeutic transgene production is monitored by indirectly by enzymatic assay for SEAP activity.

Other viral vectors may be employed as expression vectors in the present invention. Vectors derived from viruses such as vaccinia virus, adeno-associated virus (AAV), lentivirus, and herpes viruses may be employed.

Several non-viral methods, such as physical methods, chemical methods and other biochemical methods for the transfer of expression constructs into mammalian cells may also be employed in the present invention. Examples of these methods include, but are not limited to calcium phosphate precipitation, electroporation, activated-dendrimer, direct microinjection, DNA-loaded liposomes and lipofectamine-DNA complexes, cell sonication, gene bombardment using high velocity microprojectiles, and receptor-mediated transfection.

Alternatively, the expression vector may simply consist of naked recombinant DNA or plasmids. Transfer of the construct may be performed by any of the methods mentioned above which physically or chemically permeabilize the cell membrane.

Construction and Characterization of Reconstituted Amniotic Membranes.

Preparation of reconstituted amniotic membranes. The freshly isolated amniotic membranes will be sterilized by lethal dose of gamma irradiation. Alternatively, the freshly isolated amniotic membrane will be sterilized by nucleic acid cross-linking reagents, preferably, Cerus Corporation's Helinx® compounds followed by UV radiation. The treated amniotic membranes will be stripped off of the epithelial cells by incubating with 8 mM EDTA/EGTA in PBS for two hour at 37° C., followed by mechanical scraping with cell scrapers [16]. Such a sterilized amniotic membrane can then serve as a native substrate for cultured amniotic epithelial cells. The cultured amniotic epithelial cells are transfected or transduced with expression vectors exogenous to the epithelial cells and the membrane. The inventor believes that these reconstituted amniotic membranes should maintain the specific property of the amniotic epithelial cells, meanwhile minimizing the risk of potential pathogen-cross contamination. The resulting amniotic membrane will be washed in culture medium and then placed on a hydrophobic surface with basement membrane facing up. Cultured human amniotic epithelial (HAE) cell or its derived cell lines will be resuspended and seeded onto the denuded amniotic membrane in a small volume. The epithelial cells will be confined to a small area over the amniotic membrane by the hydrophobic surface to achieve a high seeding density. A subset of denuded amniotic membranes will be coated with matrix protein laminin and fibronectin before seeding of epithelial cells. After cell attachment, the reconstituted amniotic membrane will be transferred to a new dish and cultured until the epithelial cells reach confluency. The culture media will be supplemented with extra lipids, which may prolong the survival of HAE cells in a nutrient-poor environment.

Verification of the Efficacy and Safety of Reconstituted Amniotic Membrane in a Rabbit Ear Ischemic Model.

Creation of dermal wound on ischemic rabbit ear. The ischemic dermal wound will be created by ligating the rostral and central arteries of rabbit ears following a published method [17]. Four 6-mm diameter dermal wounds to the depth of bare cartilage will be created by a surgical punch biopsy instrument. The amniotic membranes will be carefully placed onto the wound sites with epithelial cells facing down toward the wound site. The dosage of growth factors will be adjusted by the size of transplanted amniotic membrane cut with appropriate size trephines. Wound sites will be protected by a transparent polyurethane film wound dressing, Tegaderm.

Determination of kinetics of transgene concentration at wound sites in vivo. The study will be conducted in both ischemic and nonischemic wound sites on rabbit ears. The viability of the amniotic membrane will be monitored by continuous GFP signals under a fluorescence microscope. The wound exudates will be harvested with a glass capillary every 24 hours for 8 days. In order to collect the wound exudates on a more consistent level, 50 microliters of PBS will be applied to each wound site 5 minutes before the wound exudates are collected with the glass capillary. A new Tegaderm film will be reapplied after each wound exudate sample collection. The transgene products concentration in wound exudates will be assayed by monitoring SEAP enzyme activities by chemiluminescent technique. The profile of SEAP activity over the experimental period will likely parallel of that of secretory growth factor.

Isolation of HAE cells are accomplished by trypsin digestion [18]. HAE cells are genetically modified to express two marker proteins, GFP and SEAP, through retroviral vectors or transfection of plasmid DNA. The cytosolic marker, GFP, will be lost if HAE cell is damaged or sloughed off from the reconstituted amniotic membrane. The secreted marker, SEAP, will be used to quantify the transgene production by the reconstituted amniotic membrane. The secreted SEAP in the reconstituted amniotic membrane culture medium can be quantified by chemiluminescent assay.

A newly developed immortalizing technique based on human telomerase reverse transcriptase (hTERT) can avoid such potential adverse effects associated with SV40 T antigen [19-21]. hTERT, which is normally expressed only in embryonic stem cells, germline cells, and cancer cells, maintains the length of the telomeres, the DNA repeats at the end of chromosomes. The constitutively expressed hTERT will be able to maintain the length of telomeres in cell lines. The expression of hTERT will not alter the cell cycle pathways as SV40 T antigen does. Therefore, telomerase immortalized cells will not exhibit the transformed phenotype. The expression of hTERT, in primary cultured HAE cells or corneal and conjunctival epithelial cells through retroviral vectors will be used to establish immortalized cell lines. The immortalized HAE cells will be further modified to express two marker proteins, GFP and SEAP, through retroviral vectors.

A successful wound healing may accidentally entrap some HAE cells at the wound sites. To ensure that immortalized HAE cells will not become tumors in the host, we will further genetically modify the HAE to express suicide protein, Herpes Simplex Virus thymidine kinase (HSV-tk). Entrapped HSV-tk positive cells at wound site can be destroyed by administering prodrug ganciclovir, which will further ensure the safety of reconstituted amniotic membrane in clinical application. The growth factor PDGF-β will be selected to be expressed by HAE cells as a therapeutic protein to promote healing of ischemic wounds.

By genetically modifying human amniotic membrane cells, the genetically modified amniotic membrane is converted into a “live factory” or a “bioreactor”, which can manufacture and deliver therapeutic proteins in situ at the wound site.

Furthermore, owing to the versatility and exchangeability of the isolated membrane, the transgene production and delivery can be sustained for a long duration by simply replacing a used amniotic membrane with freshly prepared amniotic membrane when necessary. Therefore, this approach may overcome the obstacles encountered in conventional gene therapy approaches, such as short duration of transgene expression, and host immune response to the vector or the transgene itself.

In addition, the amniotic cells can be transduced by several expression vectors encoding for different therapeutic proteins simultaneously. This could make a combinational therapy a relatively easy task. Compared with many previous attempts, the novel biomaterials of the present invention can provide a sufficient quantity of therapeutic proteins for a sufficient length of times to effectively promote healing for chronic wounds.

The preferred adenoviral vector will encode two transgene units, one encoding for a reporter protein, the secreted form of human placental alkaline phosphatase (SEAP), the other encoding for specific therapeutic protein. SEAP activity can be monitored through a highly sensitive chemiluminescent assay. The assay can detect as little as 10⁻¹³ g of SEAP protein, and remains linear over a 10,000 fold range of protein concentration. The amount therapeutic transgene production would be monitored by indirectly by enzymatic assay for SEAP activity.

Other Viral Vectors

Other viral vectors may be employed as expression vectors in the present invention. Vectors derived from viruses such as vaccinia virus, adeno-associated virus (AAV), lentivirus, and herpes viruses may be employed.

Non-Viral Methods for Gene Transfer

Several non-viral methods for the transfer of expression constructs into mammalian cells may also be employed in the present invention. Examples of non-viral methods include, but are not limited to calcium phosphate precipitation, electroporation, activated-dendrimer, direct microinjection, DNA-loaded liposomes and lipofectamine-DNA complexes, cell sonication, gene bombardment using high velocity microprojectiles, and receptor-mediated transfection.

Alternatively, the expression vector may simply consist of naked recombinant DNA or plasmids. Transfer of the construct may be performed by any of the methods mentioned above which physically or chemically permeabilize the cell membrane.

### EXAMPLES

### Example 1

### Transduction of Human Amniotic Membrane by Recombinant Adenoviral Vector

Recombinant adenoviruses have been used extensively as a highly efficient gene transfer vector. In the present invention, it is demonstrated that the first-generation replication-deficient recombinant adenovirus, AdGFP, can transfer a reporter gene GFP into human amniotic epithelial and mesenchymal cells in situ with high efficiency.

a) Construction of Recombinant Adenoviral Vector

Replication-deficient recombinant adenoviral vectors are constructed following published methods (He, T. C., Zhou, S., da Costa, L. T., Yu, J., Kinzler, K. W., Vogelstein, B. (1998) A simplified system for generating recombinant adenoviruses. Proc Natl Acad Sci USA, 95, 2509-14). The transgene is under a cytomegalovirus (CMV) promoter control and a marker protein, green fluorescent protein (GFP) or SEAP, is under a separate CMV promoter control in the same adenoviral construct. Incorporation of GFP or SEAP into an adenoviral vector not only serves as a quality control for adenoviral transduction efficiency, but also as an indicator for survival of transduced cells. All the recombinant adenoviruses are verified by PCR methods. In addition, the adenoviral vectors are further assessed by Western blotting, immunocytochemistry or functional assay after transduction into mammalian cells.

b) Preparation of Human Amniotic Membrane.

In an exemplary experiment, full-term human placenta and fetal membranes were acquired from the UCSF Birth Center according to protocols approved by the UCSF Committee on Human Research. The amniotic membranes were harvested by mechanically separating them from the placenta and other fetal membranes. The separated amniotic membranes were thoroughly washed, first with PBS and then PBS containing penicillin (100 U/ml) and streptomycin (100 mg/ml) to remove blood clots and other contaminants (Kim, J. C., Tseng, S.C. (1995) Transplantation of preserved human amniotic membrane for surface reconstruction in severely damaged rabbit corneas. Cornea, 14, 473-84.). The harvested amniotic membrane was cut into 1.5×1.5 cm² pieces and placed in culture dishes and maintained in PBS supplemented with 1% bovine serum albumin (BSA).

c) Transduction of Human Amniotic Membrane.

Generally, human amniotic membrane may be pretreated with 1 ml of 8 mM of Ethyleneglycol-bis-(b-amino ethyl ether) N,N,N′,N′-tetraacetic Acid (EGTA) in Dulbecco's 8 modification of Eagle's basal medium (DMEM) for 30 minutes at 37° C. About 2.5×10⁸ plaque forming units (pfu) recombinant adenovirus is added directly into the culture dish containing a single 1.5×1.5 cm² of amniotic membrane segment. After two hours incubation at 37° C., culture medium is removed and replaced by 2 ml of fresh DMEM supplemented with 5% fetal bovine serum (FBS). The transduction efficiency is monitored by examining GFP expression under the fluorescent microscope 24 hours after the transduction.

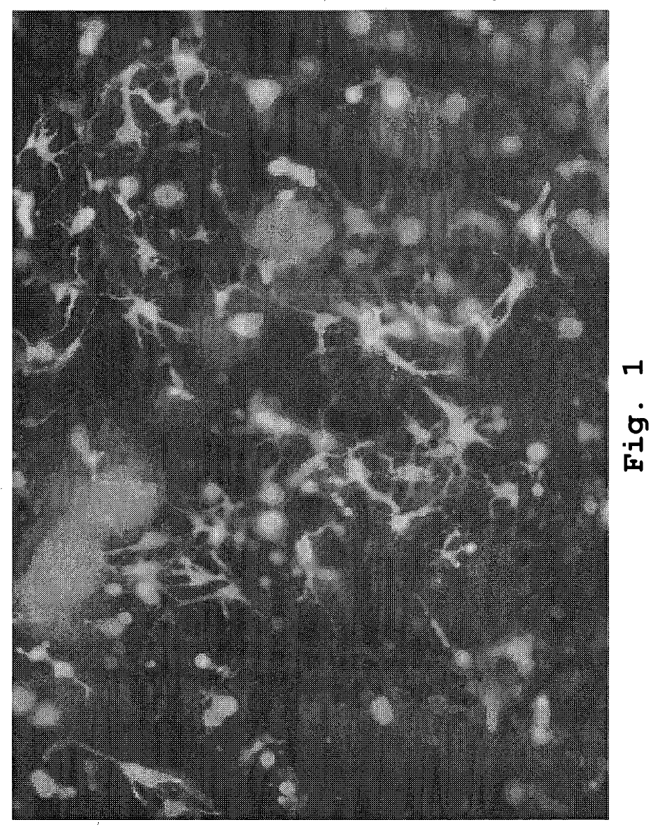

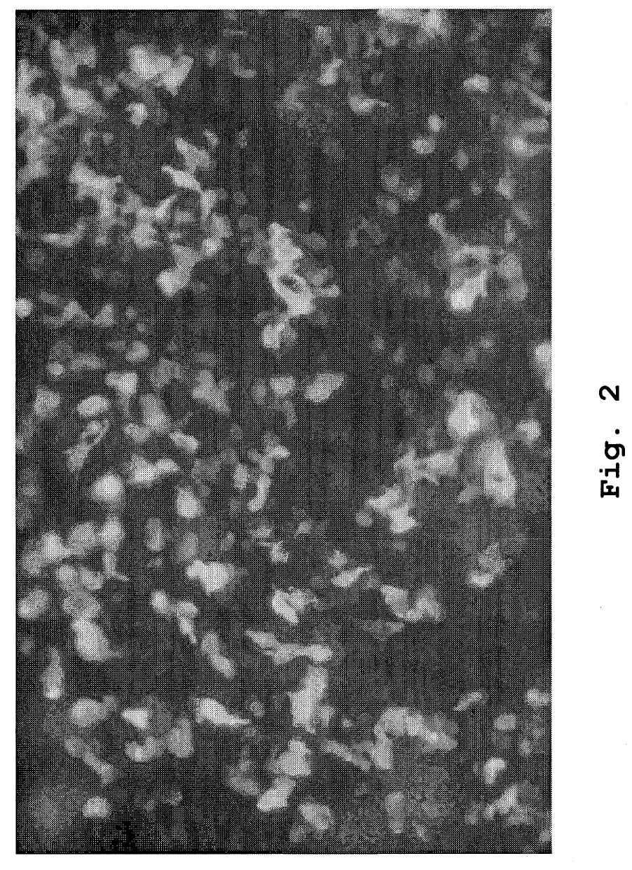

In an exemplary experiment, adenoviral transduction was carried out by adding about 2.5×10⁸ pfu AdGFP into a culture dish containing a single 1.5×1.5 cm² of amniotic membrane segment. The expression of GFP was examined under the fluorescent microscope at 24 hours post-transduction. Strong GFP expression was observed in amniotic mesenchymal cells in every amniotic membrane preparation (FIG. 1). However, the adenoviral vector transduced epithelial cells with high efficiency (>60%) in only about 20% of amniotic membrane preparations (FIG. 2). There was no visible cytotoxicity to amniotic cells after the adenoviral transduction at the adenoviral titer used. GFP expression was still observed after four weeks of organ culture (DMEM/5% FBS) without significant reduction in marker protein GFP expression. Therefore, amniotic cells can be transduced by the adenoviral vector with high efficiency. Transgene expression is sustained for at least four weeks in organ culture conditions.

### Example 2

### Human Amniotic Membrane can be Transduced by Adenoviral Vector

Recombinant adenoviruses have been used extensively as a highly efficient gene transfer vector. We have demonstrated that a replication-deficient recombinant adenovirus (AdGFP) can transfer a reporter gene, green fluorescent protein (GFP) [23], into human amniotic epithelial (HAE) and mesenchymal cells in situ with high efficiency. The amniotic membranes were harvested by mechanically separating them from the placenta and other fetal membranes. Adenoviral transduction was carried out by adding about 2.5×10⁸ pfu AdGFP into a culture dish containing a single 1.5×1.5 cm square of amniotic membrane segment (about 150 viruses per cell). Strong GFP expression was observed in amniotic mesenchymal cells in every amniotic membrane preparation (FIG. 1).

However, the adenoviral vector transduced HAE cells with high efficiency (>60%) in only about 20% of amniotic membrane preparations (FIG. 2). We have found that by including 8 mM of EGTA in culture media to disrupt tight junctions of HAE cells during infection, we can significantly enhance adenoviral transduction efficiency of the cells.

### Example 3

### Improved Transduction of Amniotic Epithelial Cells by EGTA Treatment

Amniotic epithelial cells outnumber mesenchymal cells about 10 to 1 in the full-term amniotic membrane (Casey, M. L., MacDonald, P. C. (1996) Interstitial collagen synthesis and processing in human amnion: a property of the mesenchymal cells. Biol Reprod, 55, 1253-60.). Therefore, transduced amniotic epithelial cells could potentially produce much more transgene product than could amniotic mesenchymal cells. Transgene products produced by amniotic epithelial cells can access the wound site without having to diffuse through the matrix of the amniotic membrane. In addition, it will be shown below that adenoviral-transduced amniotic epithelial cells can survive much longer in a nutrient-poor environment than can mesenchymal cells. However, fewer than 20% of the amniotic membrane preparations exhibit a high efficiency (>60%) of adenoviral transduction in amniotic epithelial cells. It would be desirable to enhance the transduction efficiency of amniotic epithelial cells through biological manipulation.

It has been reported that the distribution of adenoviral receptors on the basolateral domain of airway epithelial cells accounts for its low transduction efficiency by the adenoviral vectors (Walters, R. W., Grunst, T., Bergelson, J. M., Finberg, R. W., Welsh, M. J., Zabner, J. (1999) Basolateral localization of fiber receptors limits adenovirus infection from the apical surface of airway epithelia. J Biol Chem, 274, 10219-26.). The reagents that disrupt the tight junction of airway epithelial cells, such as Ethyleneglycol-bis-(?-amino ethyl ether) N,N,N′,N′-tetraacetic Acid (EGTA), can enhance the transduction efficiency of airway epithelial cells by adenoviral vectors.

In the present invention, 8 mM of EGTA in DMEM is used to disrupt tight junction of amniotic epithelial cells by 2.5 hours incubation at 37° C., which leads to a drastic enhancement of adenoviral transduction efficiency of amniotic epithelial cells. It remains to be seen whether there is a basolateral distribution of adenoviral receptors on amniotic epithelial cells which might be responsible for the low transduction efficiency of amniotic epithelial cells in 80% of untreated amniotic membrane. However, significant improvement of transduction efficiency of amniotic epithelial cells is shown here.

### Example 4

### Survival of Recombinant Amniotic Membrane in Nutrient-Poor Environment

The protein delivery system mediated by a recombinant tissue membrane may be applied to dermal surface or ocular surface gene therapy. In either case, the amniotic membrane may be subjected to a nutrient-poor environment. Although the wound exudate could provide a limited source of nutrients, the dermal surface is generally considered to be a nutrient-poor environment, especially in the setting of ischemia and concomitant infection. Tears represent a nutrient-poor environment for the amniotic membrane, with almost no carbohydrate and about 1.5% of various soluble proteins (Van Haeringen, N. J. (1981) Clinical biochemistry of tears. Surv Ophthalmol, 26, 84-96). It would be critically important for the successful application of this protein delivery system to the dermal or ocular surface gene therapies that the adenovirus-transduced amniotic membrane can survive in a nutrient-poor environment.

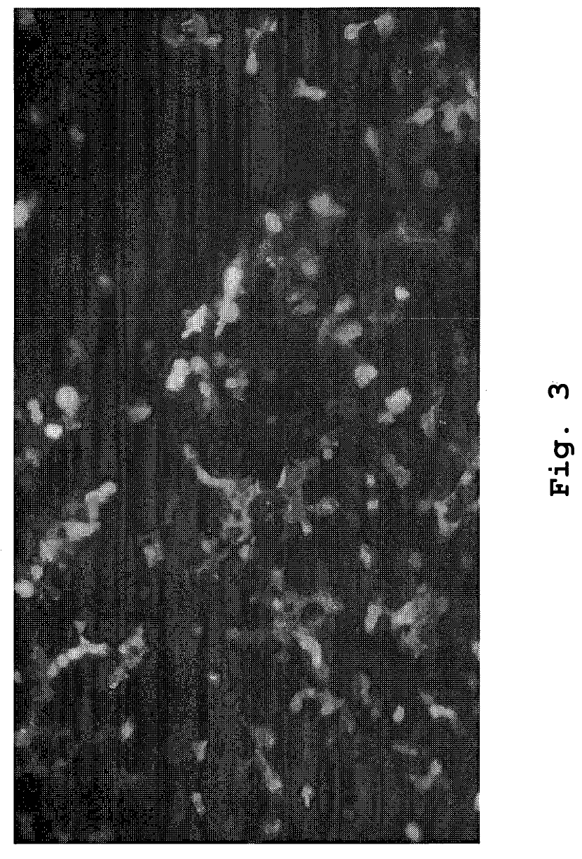

In this study, we have demonstrated that HAE cells can survive in a nutrient-poor medium and continuously produce transgene product for more than three weeks. PBS supplement with 1.5% bovine serum albumin (BSA) was used to simulate tears as a nutrient-poor medium for amniotic membrane culture. The amniotic membrane was transduced with AdGFP as described above. The amniotic membrane was transferred to a nutrient-poor medium 48 hours after transduction. The culture media was changed every two days throughout the experiment. The amniotic membrane was photographed every day for the first week. Then it was photographed once a week. Both adenoviral-transduced amniotic epithelial and adenoviral-transduced mesenchymal cells can survive well in a nutrient-poor environment for the first 72 hours. The GFP signal in amniotic mesenchymal cells significantly reduced on the fourth day in a nutrient-poor medium in contrast to four weeks in normal culture medium. On the other hand, the GFP signal in amniotic epithelial cells persisted for three weeks even in a nutrient-poor medium (FIG. 3).

This suggests that HAE cells can survive in a nutrient-poor environment for more than three weeks after adenoviral transduction. However, by the fourth week, the GFP signal was reduced and the HAE cells begin to assume a round morphology instead of the normal polygonal morphology.

### Example 5

### Adenoviral-Transduced Amniotic Epithelial Cells Can Continuously Produce Transgene Product in a Nutrient-Poor Medium

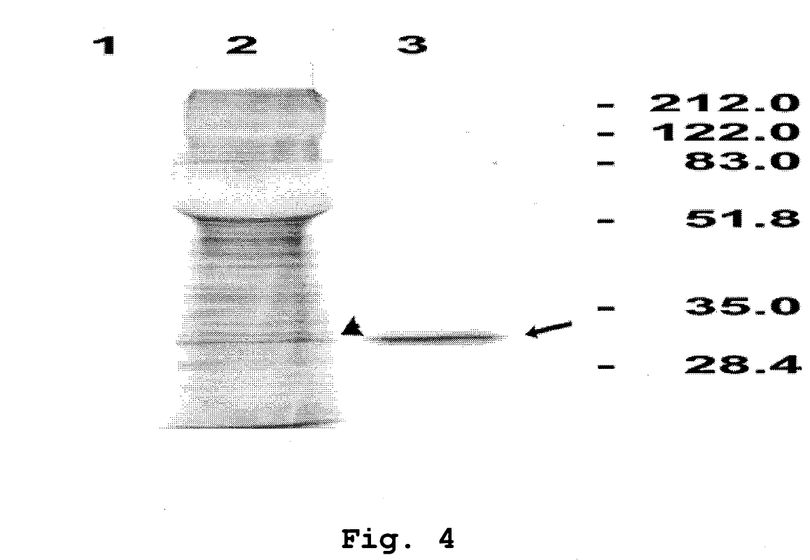

In order to further confirm that HAE cells will not only survive but also continuously produce transgene product GFP, we conducted a radioactive metabolic labeling experiment by incubating HAE cells with ³⁵S-cysteine and ³⁵S-methionine. The amniotic membrane was transduced with AdGFP as described above. The adenoviral-transduced amniotic membrane was transferred to a nutrient-poor medium 48 hours after transduction and cultured for 6 days. The culture media was changed every two days throughout of the experiment. The adenoviral-transduced amniotic membrane was then metabolic labeled at end of day 5 (most of GFP signals in mesenchymal cells disappeared by that time) with 2 mCi of ³⁵S-cysteine and ³⁵S-methionine in 2 ml PBS/1.5% BSA for 12 hours. The amniotic membrane was repeatedly washed in cold PBS and the amniotic cells were lysed with 1% Triton X-100 in Tris-HCl buffer. Half of cell lysate was immunoprecipitated with monoclonal antibody against GFP (Quantum Biotechnologies, Inc., Montreal, Canada). Both total cell lysate (lane 2) and immunoprecipitated GFP (lane 3) were analyzed on SDS-PAGE (FIG. 4).

There was a strong metabolic labeling of GFP in the HAE cells. The GFP is one of the most prominent bands in the whole cell lysate lane, which suggests that the GFP is the most actively produced protein in HAE cells in nutrient-poor medium. Our preliminary study also suggested that other proteins, such as PDGF-β and the secreted form of human placental alkaline phosphatase (SEAP) [24], can also be produced by adenoviral-transduced HAE cells in a nutrient-poor medium.

This observation is not surprising considering the facts that amniotic membrane is an avascular tissue, and the amniotic epithelial cells are bathed in a relatively nutrient-poor amniotic fluid in the womb. Thus, the amniotic epithelial cells may be able to adopt special metabolic pathways to survive in a nutrient-poor environment. In fact, many amniotic epithelial cells have large amounts of lipid droplets in their cytosol and a high expression level of low-density lipoprotein (LDL) receptors [25]. It is reasonable to speculate that these lipid deposits may, in part, contribute to the energy supply for amniotic epithelial cells in a nutrient-poor environment. These preliminary studies suggest that the amniotic membrane, with its own power supply, has the potential to become a useful protein delivery transplant in a nutrient-poor environment.

### Example 6

### MDCK Cells Seeded Onto Amniotic Membrane Can Assume High Density and Survive for Extended Periods in a Nutrient-Poor Medium

Due to its fast growth rate and well-defined epithelial morphology, cell line Madin-Darby canine kidney (MDCK) cells was chosen for constructing prototypic reconstituted amniotic membrane in our pilot experiment. The epithelial cell line Madin-Darby canine kidney (MDCK) cells has been doubly-transduced with retroviral vectors encoding for marker proteins GFP and SEAP. The transduced MDCK cells were seeded on a denuded human amniotic membrane at high density. After confluency, the transduced MDCK cell density can reach a level similar to that of HAE cells on native amniotic membrane. That is about 2 to 3 times the typical cell density that transduced MDCK can reach on the plastic substrate. Interestingly, the transduced MDCK cells seeded on amniotic membrane can survive in PBS/1.5% BSA medium for about 5 days, about 2 days longer than when it is seeded on plastic flask. It is not clear what is the underlying mechanism for the increased fitness of transduced MDCK cells seeded on amniotic membrane.

### Example 7

### Delivery of Therapeutic Proteins to Wounds with Infection and Ischemia

Infection and hyperactive inflammation are commonly associated with chronic ulcers. According to the present invention, therapeutic proteins, including a group of antimicrobial proteins and anti-inflammatory proteins can be expressed and delivered by the recombinant tissue membrane such as adenoviral-transduced amniotic membranes for healing of bacterial infected wounds.

a) Creation of Dermal Wound on Ischemic Rabbit Ear

Dermal ulcer wound healing on ischemic rabbit ear is a well established wound healing model (Ahn, S. T., Mustoe, T. A. (1990) Effects of ischemia on ulcer wound healing: a new model in the rabbit ear. Ann Plast Surg, 24, 17-23). In this model, healing occurs almost entirely by formation of new granulation and epithelial tissue. The ischemic dermal wound is created by ligating the rostral and central arteries of rabbit ear following a published method (Ahn, S. T., Mustoe, T. A. (1990) Effects of ischemia on ulcer wound healing: a new model in the rabbit ear. Ann Plast Surg, 24, 17-23). Five 6-mm diameter dermal wounds to the depth of bare cartilage will be created by a surgical punch biopsy instrument. The amniotic membranes is carefully placed onto the wound sites, with epithelial cells facing the wound. Wound sites is protected by a transparent polyurethane film wound dressing (Tegaderm, 3M, Minneapolis, Minn.).

b) Determination of Kinetics of Transgene Production by Adenoviral-Transduced Amniotic Membrane In Vitro

The kinetics of transgene production by adenoviral-transduced amniotic membrane can be addressed by an adenoviral vector encoding for both the secreted form of human placental alkaline phosphatase (SEAP) and GFP (AdSEAP/GFP). It has been reported that SEAP is efficiently secreted from transduced cells, and the levels of SEAP activity in the culture medium are directly proportional to changes in intracellular concentrations of SEAP mRNA and protein (20, 21). The marker protein, SEAP, can be conveniently assayed by a highly sensitive chemiluminescent technique. The SEAP secreted by a live amniotic membrane may serve as a surrogate marker for secretory transgenic proteins in the determination of kinetics of transgene production and release by the adenoviral-transduced amniotic membrane both in vivo and in vitro.

The in vitro kinetics studies include the amniotic membrane maintained in both in nutrient-poor (PBS with 1.5% BSA) media and in nutrient-rich (DMEM with 5% FBS) media as a control. Although the wound exudate could provide a limited source of nutrients, the dermal surface is generally considered to be a nutrient-poor environment, especially with ischemic condition and concomitant infection. The in vitro study in a nutrient-poor medium is considered to reflect the kinetics of transgene production and release closer to the condition will be encountered in chronic ulcers.

A group of freshly prepared human amniotic membranes (12 independent specimens, within 24 hours of preparation) will be transduced with a recombinant adenoviral vector AdSEAP/GFP, encoding for both marker proteins GFP and SEAP, in vitro. The transduced amniotic membranes will be maintained in 2 ml of either nutrient-rich (6 specimens) or nutrient-poor (6 specimens) media. The viability of the amniotic membrane will be monitored by continuous GFP signals. The culture media will be replaced every 24 hours, the spent media will be used for SEAP enzymatic assays by chemiluminescent technique. The resulting enzymatic activity at each time point will be normalized with the enzymatic activity detected at posttransduction 24 hours. The SEAP activity of each individual amniotic membrane will be monitored for 21 days. This experiment will provide us the information regarding transgene production in both media over the experimental periods.

Transgene product concentration at the wound site is determined by both the production and proteolytic destruction of transgene product at the wound site. The production and secretion of transgene product by the amniotic membrane could be influenced by the multiplicity of infection (moi) of adenoviral virus, the size of amniotic membrane segment and the nutritional condition at the wound site. The stability of transgene product at wound site may be dependent on the activity and amount of proteolytic enzymes available at the wound site. It is expected that different type wounds may have different nutritional condition and different amount of proteolytic enzymes available. Therefore, the kinetics of transgene concentration at wound site is studied in both normal rabbit ear wound and ischemic rabbit ear wound models.

### Example 8

### PDGF-β Delivered by Prototypic Reconstituted Amniotic Membrane can Promote Healing of Ischemic Wounds on Rabbit Ears

Growth factor PDGF-β, the only FDA approved protein-drug for the treatment of chronic wounds, was selected as the protein-drug to be delivered by the reconstituted amniotic membrane. MDCK cells have been triple-transduced with retroviral vectors encoding for marker proteins GFP, SEAP, and PDGF-β. Expression of all transgenes was controlled by cytomegalovirus (CMV) promoter element, and SEAP was included for quantitation of secreted protein production by the reconstituted amniotic membrane. The control amniotic membrane seeded with transduced MDCK cells expresses only GFP and SEAP.

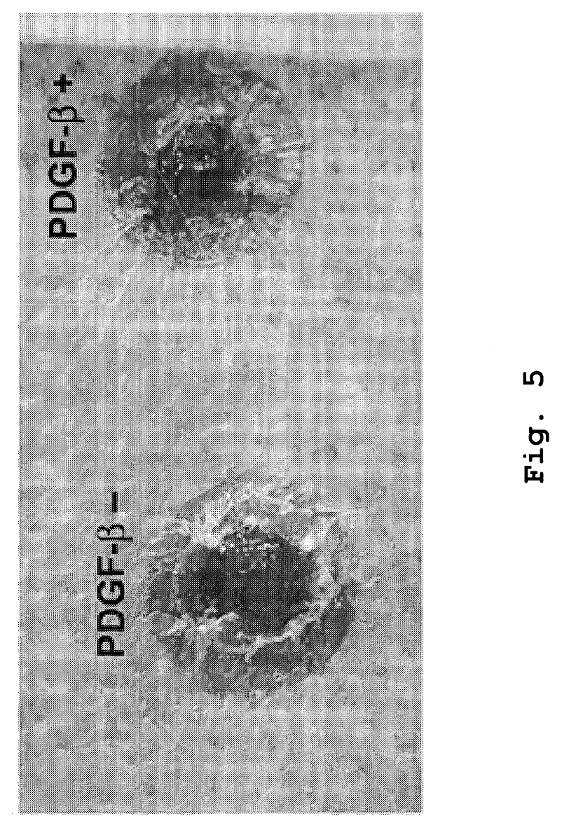

We choose dermal wound healing on ischemic rabbit ear to simulate the ischemic conditions of human chronic wounds. Due to a lack of loose connective tissue between the skin and cartilage, wound healing in this model occurs almost entirely by formation of new granulation and epithelial tissue. The ischemic dermal wound was created by ligating the rostral and central arteries of rabbit ears following a published method [17]. The opposite ear was used as nonischemic control. Four 6-mm diameter dermal wounds to the depth of bare cartilage were created by a surgical punch biopsy instrument on each ear. A 6 mm diameter reconstituted amniotic membrane was placed at each rabbit wound site with epithelial cells facing down. The wound sites were further protected by a transparent polyurethane film wound dressing, Tegaderm. The wound sites were examined every other day and monitored by photography. Healing of ischemic wounds is severely retarded compared to normal wounds. The wounds treated with MDCK-PDGF heal consistently faster than the control wounds on both ischemic and control ears, up to 3-fold reduction of wound area at 16 days after surgery (FIG. 5).

When examined at the wound site under the fluorescence microscope, there was no visible loss of GFP signal during the first week of examination. Significant loss of GFP fluorescence was noticed after about 9 to 10 days, which may be attributed to host immune response to the MDCK-PDGF cells. In conclusion, we found that the reconstituted amniotic membrane, delivering growth factor PDGF-β, is effective in promoting healing of ischemic wounds on rabbit ears.

Transplantation of AdGFP-transduced amniotic membrane was applied onto rat corneal surface. The AdGFP-transduced amniotic membrane was examined under the fluorescent microscope 2 days after viral transduction to verify efficient transduction of amniotic cells. Recipient rats were anesthetized prior to the procedure. The transduced amniotic membrane was rinsed with PBS and trimmed into proper size to cover an area slightly larger than the corneal surface. The amniotic membrane, epithelial side outward, was sutured to the conjunctiva by four interrupted sutures (10-0 nylon). The transplant was protected with a tarsorrhaphy.

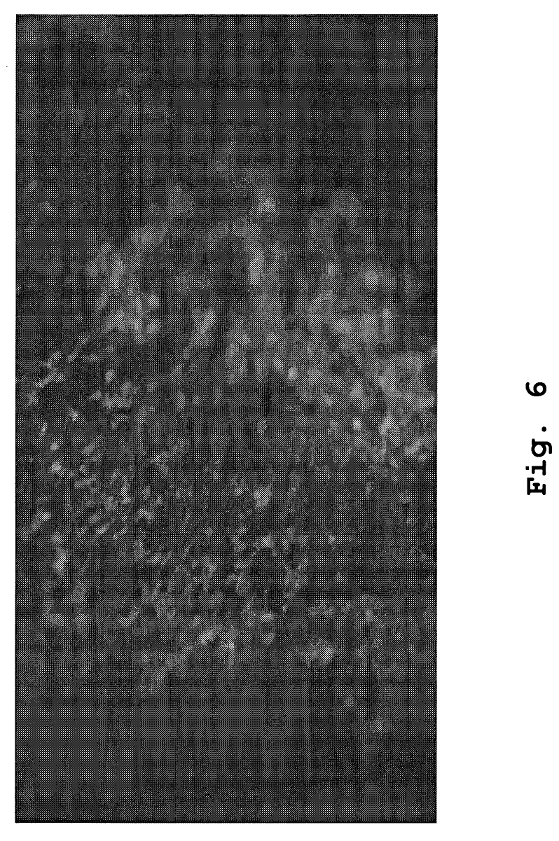

The same rat was examined under a modified fluorescent microscope 24, 48 and 72 hours after transplantation. GFP signal in amniotic cells was observed at the 24 (FIG. 6) and 48 hour time point but not at the 72 hour time point. Since the GFP expression was observed to last for more than a week in previous organ culture experiments, we hypothesize that GFP transduced amniotic cells may have died either through apoptosis or necrosis. The loss of GFP signal was accelerated by the desiccation of the amniotic membrane (due to partially failed tarsorrhaphy) on the ocular surface.

The study of kinetics of transgene concentration at wound site is conducted in both types of wound sites on rabbit ears (8 rabbits each group). The human amniotic membrane is transduced with a recombinant adenoviral vector encoding for both marker proteins GFP and SEAP in vitro. The viability of the amniotic membrane is monitored by continuous GFP signals under a fluorescent microscope. The wound exudates are harvested with a glass capillary every twenty-four hours for 8 days. In order to collect the wound exudates on a more consistent level, fifty microliter of PBS is applied to each wound sites 5 minutes before the wound exudate are collected with glass capillary. The transgene products concentration in wound exudates are assayed by monitoring SEAP enzyme activities by chemiluminescent technique. The profile of SEAP activity over the experimental period should parallel of that of secretory therapeutic proteins in the subsequent aims. The ability of the transduced amniotic membrane to continuously release growth factors or cytokines at the wound site can make the usage of live amniotic membrane feasible.

### Example 9

### Sustained Presence of Therapeutic Proteins on Healing of Acute Wounds

Treatment of chronic ulcers frequently involve surgical debridement to convert the chronic ulcers into an acute wound. The main events of acute wound healing process include proliferation, migration and differentiation of fibroblast and endothelial cells. According to the present invention, a recombinant amniotic membrane can be applied to the wound site and deliver therapeutic protein expressed by the genetically modified cells in the membrane, thereby promoting the wound healing process.

The recombinant amniotic membrane applied onto the wound site provides an unique environment where sustained, and perhaps, excessive supply of growth factors will be encountered at the wound site. Various therapeutic proteins, including growth factors, anti-inflammatory proteins and angiogenic factors, can be delivered by adenoviral-transduced amniotic membranes in promoting wound healing.

Two uncomplicated wound healing models, a full thickness wound model on rabbit ear and a partial thickness wound model on guinea pig, are used to address the wound healing process commonly encountered in surgical wound healing or at the skin donor site. It is desirable that wound healing speed is accelerated and scar tissue formation is reduced. A group of growth factors, such as platelet-derived growth factor (PDGF) and transforming growth factor-b (TGF-b), and anti-inflammatory proteins, such as interleukin-10 (IL-10) and interleukin-1 receptor antagonist (IL-1ra), are the bioactive molecules expressed by the recombinant amniotic membrane.

a) Expression of GFP by Amniotic Membrane Applied onto Rat Dermal Wound Sites

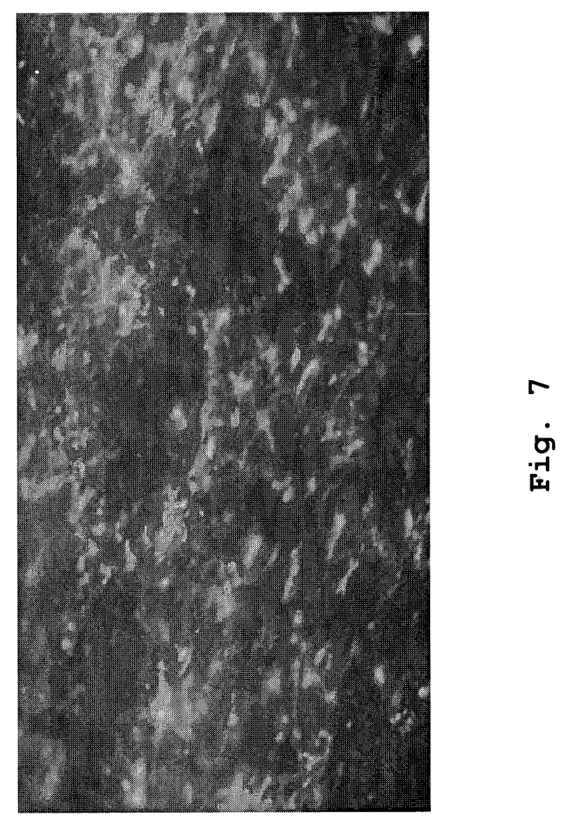

In an exemplary experiment as proof of principle, AdGFP-transduced amniotic membrane which expresses a reporter protein, GFP, is applied onto rat dermal wound sites. Recipient rats were anesthetized prior to the procedure. Acute incisional full thickness dermal wounds were surgically created on the back of female Sprague Dawley rats. The AdGFP-transduced amniotic membranes were examined under the fluorescent microscope four days after adenoviral transduction to verify efficient transduction of amniotic cells. The transduced amniotic membranes were rinsed with PBS and trimmed into proper size to cover the dermal wound sites. The AdGFP-transduced amniotic membranes were secured by suturing to the subcutaneous tissue by four interrupted sutures (8-0 silk). The wound sites and the amniotic membranes were protected by Tegaderm film wound dressing. The same rat was examined under a modified fluorescent microscope daily for 7 days after application of amniotic membrane. GFP signals observed in amniotic cells were compatible with those observed in normal culture medium (DMEM/10% FBS) for the first three days (FIG. 7). The GFP signal gradually reduced after the end of day four, which might related to the wound infection, but was still clearly visible by the end of day seven. These results demonstrate that adenoviral-transduced amniotic membrane can be used for long term protein delivery at dermal wound sites.

b) Delivery of Growth Factors by Recombinant Amniotic Membrane

The growth factors has been intensively studied in promoting wound healing in the past decade. All the published results mostly are dealing with the purified growth factors in topical application. The amniotic membrane-mediated protein delivery system can provide a wound environment with continuous supply of growth factors or other therapeutic proteins. Two growth factors, PDGF-bb and Transforming growth factor (TGF)-b, on dermal wound healing, have proven clinical efficacy and broad spectrum of effects on target cells. Other growth factors such as FGF-1, FGF-2, KGF, KGF-2, TGF-b, VEGF, epidermal growth factor (EGF), TGF-a and epiregulin, may also be used individually or in combination. Even though most growth factors, with the exception of PDGF-bb (3, 25-27), have not produced significant improvement in clinical trials (28), most of these growth factors have been shown to be effective in promoting dermal wound healing in animal models by topical application (29-36).

cDNAs encoding growth factors, anti-inflammatory and antimicrobial proteins can be produced by RT-PCR from human placenta mRNA. The RT-PCR products are cloned and sequenced before being used for construction of recombinant adenoviral vectors, recombinant retroviral vectors, and plasmid vectors.

Each segment of amniotic membrane are co-transduced by two adenoviruses, each encoding for an intended therapeutic protein and marker protein SEAP, respectively, at 10 to 1 ratio. The amount of transgenic product produced by the adenoviral-transduced amniotic membrane can be quantitated indirectly by measuring the SEAP enzymatic activity in culture media secreted by the adenoviral-transduced amniotic membrane. The amount of transgenic products delivered to the wound site are standardized by adjusting the size of the adenoviral-transduced amniotic membrane segment.

The most significant wound healing process is the granulation, therefore, the dermal fibroblast growth and neovascularization process are the important steps for wound healing process. Therefore, before the rabbit ear wound healing experiments are carried out, the wound closure of cultured human foreskin fibroblast cells and human umbilic cord vein endothelial cells (HUVEC) may be studied in order to provide the vital information on the optimal dosage and duration of growth factors to be used in the in vivo applications.

Wound closure of cultured cells reflects both cell mitotic activity and cell migration, both those parameters will be examined in this study. The growth factor is delivered by amniotic membranes with an equivalent amount of SEAP activity, which is placed into each culture dish with human foreskin fibroblast cells or HUVEC. The dosage of growth factors is controlled by the size of amniotic membrane segments and the duration of amniotic membrane presence in the culture media. Several different dosages of growth factors (8 dishes of cells for each dosage) are tested for the optimal range of growth factor concentration for use in the in vitro wound closure model. The changes in wound closure rate and mitotic activity upon growth factor addition are monitored by image processing and bromodeoxyuridine (BrdU) staining. The data collected from these studies are applied to a better design for in vitro and in vivo study of amniotic membrane-mediated growth factor delivery.

An in vitro wound closure model is used to study the effects of growth factor application by scratch wounds on tissue cultured human dermal fibroblast cells. The wound closure process is followed by photography every four hours. The photographs is digitized by a slide scanner and processed by an imaging processing software, NIH Image. The wound closure rate is plotted as percentage of wound closure over the time.

Wound healing on rabbit ear in vivo is followed by photo documentation through the transparent film dressing (Tegaderm) every two days for eight days. The amniotic membrane is placed onto the wound throughout the experimental period. The survival of amniotic cells is monitored through the presence of GFP signal by fluorescent microscopy. The final wound healing assessment is conducted at post wound Day 8 on rabbit ear. The epithelial gap, granulation tissue gap, and peak of granulation tissue is measured with a micrometer reticle using light microscopy (19). The wound healing rate is plotted as percentage of wound closure over time.

Based on the results from the in vitro study, four different dosages (8 rabbits for each dosage) are applied for in vivo animal studies for each growth factor. Special attention is paid to granulation and epithelial wound closure. Furthermore, the wound tissue fibroblast density, vascular density and inflammatory cell densities is examined histologically by the end of the experiment. Mitotic activities at the wound site is examined by in vivo BrdU labeling at mid stage (Day 4) of the wound healing process. Upon completion of each individual growth factor assay, combinations of growth factors is studied to reveal potential synergistic effects. The growth factors which promote granulation, such as TGF-b and VEGF, may be combined with epithelialization promoting factors, such TGF-a and EGF. Effects of growth factor delivered by the recombinant amniotic membrane on wound healing are compared with conventional topical application of growth factors to the wound.

c) Delivery of Therapeutic Proteins by Recombinant Amniotic Membrane

Chronic ulcers are typically accompanied by such conditions, including infection, ischemia and excessive inflammation. Therapeutic proteins, including a group of antimicrobial proteins, anti-inflammatory proteins, protease inhibitors and growth factors are be delivered by adenovirus-transduced amniotic membrane to suppress bacterial infection and control excess inflammation. The effects on wound healing are determined by using infected and ischemic dermal wound healing models on rabbit ears, and an alkaline burn model on rabbit. It is desirable that bacterial infection is suppressed; tissue inflammation is controlled; granulation is promoted; and neovascularization and wound closure results.

Chronic wounds all harbor bacteria, which greatly increase the local inflammatory reaction and promote the formation of granulation tissue. The chronic nature of many wounds is directly related to high bacterial concentrations in the wound tissues. Chronic wounds often contain abundant necrotic tissue, which is detrimental to wound healing in several ways. First, dead tissue provides a growth medium for bacteria, thus increasing the probability of wound infection. Bacteria in this environment further stimulate the prolonged elevation of pro-inflammatory mediators and cytokines. Second, necrotic tissue in a wound release endotoxins that may inhibit the migration of keratinocytes and fibroblasts into the wound. The nonviable debris also promotes a sustained inflammatory reaction, with the production of pro-inflammatory mediators and proteinases that may destroy endogenous growth factors as well as degrade the glycosaminoglycan matrix needed for wound healing. Interleukins and tumor necrosis factor, for example, promote the formation of matrix metalloproteinases and also decrease the production of tissue inhibitors of metalloproteinases (TIMP). In this environment, endogenous as well as exogenous growth factors have a decreased life span, and wound healing is impaired. Debridement of wounds reduces the bacterial load, decreases the production of inhibitors of growth factors, and may stimulate the production and release of growth factors from cells involved in healing.

The combination of long term use and insufficient tissue dosage of antibiotics provides a fertile ground for the development of antibiotic-resistant microbes, which can lead to diabetic foot ulcer patients refractory to antibiotic treatment. Topical application of high dosages of mammalian endogenous antimicrobial proteins, such as bactericidal/permeability-increasing protein (BPI), defensins and collectin, mediated by adenoviral-transduced amniotic membrane could serve as an alternative or an adjunctive therapy for controlling infections at the wound sites.

BPI has selective toxicity against Gram-negative bacteria. BPI appears to be most effective when it acts at sites of infection in synergy with defensins and the membrane attack complex of complement (4). Defensins include alpha-defensins, which are major constituents of the microbicidal granules of neutrophils, and beta-defensins, which is constitutively expressed by human epithelial cells. Defensins have wide spectra of activity directed against various bacteria, fungi, and enveloped viruses (5). Defensins and most other antimicrobial proteins act by permeabilizing the cell membranes of microorganisms. Defensins play an important role in the host defense against pathogens. In patients with cystic fibrosis, the loss of defensins activity in airway fluid leads to colonization and infection with organisms like Staphylococci and Pseudomonas (6). The mannose-binding protein (MBP) belongs to the collectin family. It selectively recognizes the carbohydrate patterns that decorate microorganisms such as bacteria, yeast, parasites, mycobacteria, and certain viruses, but not the sugars that decorate self glycoproteins (7). MBP destroys those microbes with the aid of the complement system. Mutations in the MBP gene result in recurrent infections (8, 9).

These antimicrobial proteins, including defensins, mannose-binding protein (MBP), and bactericidal/permeability-increasing protein (BPI), are used as the therapeutic protein individually and in combination there of to be delivered by the recombinant anmiotic membrane.

Additionally, anti-inflammatory proteins are also delivered to the wound site via the recombinant amniotic membrane. Inflammation is a normal step, but not necessary step, of wound healing process. However, excess of inflammation is responsible to the scar tissue formation. The Inventor believes that using appropriate amount of anti-inflammatory proteins should be able to modulate the inflammatory process and excess scar tissue formation without significantly retarding the wound closure process. Three anti-inflammatory proteins, interleukin-1 receptor antagonist (IL-1ra), interleukin-10, and soluble TNF receptor can be used as the therapeutic protein.

### Example 10

The following is an exemplary procedure for carrying out the experiments.

1) Evaluation of Bacterial Infection at Wound Site

Bacterial infection at the wound site is observed clinically for cellulitis and pus formation. A sample of wound exudate is taken for bacterial culture. The bacterial titer is determined by colony count on 7% Sheep Blood Agar (SBA) plate (PML Microbiological, Tualatin, Oreg.) after serial dilution at 4° C. The plate is incubated at 37° C. with 5% CO2 for 24 hours. By the end of experiment, the infection is further evaluated by Gram stain, and by histological evidence of neutrophil infiltration and cartilage necrosis.

2) Establishment of Bacterial Liquid Culture

All the bacterial liquid cultures is conducted in mammalian culture medium DMEM supplemented with 10% FBS. This liquid culture condition mimics the bacterial growth environment at the wound site, and also promotes survival of the adenoviral-transduced amniotic membrane which delivers antimicrobial proteins.

3) Creation of Dermal Wound on Ischemic Rabbit Ear

Dermal ulcer wound healing on ischemic rabbit ear is a well established wound healing model (19). In this model, healing occurs almost entirely by formation of new granulation and epithelial tissue. The ischemic dermal wound is created by ligating the rostral and central arteries of rabbit ear following a published method (19). Five 6-mm diameter dermal wounds to the depth of bare cartilage is created by a surgical punch biopsy instrument. The amniotic membranes is carefully placed onto the wound sites. Wound sites is protected by a transparent polyurethane film wound dressing (Tegaderm, 3M, Minneapolis, Minn.).

4) Examination of the Effect of Amniotic Membrane Expressing BPI and Defensins on P. aeruginosa and S. aureus In Vitro

The effects of overexpression of BPI and defensins is examined in liquid cultures of two strains of bacteria, Gram negative P. aeruginosa and Gram positive S. aureus. Strains from the American Type Culture Collection (ATCC, Rockville, Md.) can be used for these experiments. MBP antimicrobial function requires the complement system and therefore is examined only on an animal model. The adenoviral-transduced amniotic membrane is cultured in an antibiotic-free medium for 24 hours before the start of the experiment. The amniotic membranes with equivalent amounts of SEAP activity are placed in the wells of a multiwell plate, and various amounts of bacteria are inoculated into each well for overnight culture. The health of amniotic cells is monitored through GFP signals by fluorescent microscopy. Any damaged amniotic cells should release GFP protein into the culture medium which is no longer detectable by fluorescent microscopy. The bacterial growth is examined by turbidity of the culture medium. Furthermore, the clear wells are cultured to detect surviving bacteria. This experiment determines whether bacterial growth can be controlled by the antimicrobial proteins delivered through the amniotic membrane. In addition, this experiment also reveals the bactericidal activities of BPI and defensins, if the bacterial counts in some wells are reduced under tissue culture condition. All experiments are repeated in quadruplicate.

5) Evaluation of the Effect of Antimicrobial Proteins Delivery by Amniotic Membrane on Bacterial Infection Rate at the Wound Site

It has been reported that the infection rate is about 20% of the wound sites in the ischemic rabbit ear wound healing model (19). The amniotic membrane itself has been suggested to have antimicrobial activity through physical adherence to the wound site (38). Therefore, the infection rates in the wound sites are compared between a native amniotic membrane and a recombinant amniotic membrane that expresses antimicrobial proteins according to the present invention (8 rabbits in each group). All three groups of antimicrobial proteins are tested in this study. The infection is monitored clinically by Gram stain, selected culture of wound exudate, and histological evidence of neutrophil infiltration and cartilage necrosis. Significant reduction of infection rate by amniotic membrane alone or amniotic membrane expressing antimicrobial proteins should be helpful for designing a clinical trial for diabetic ulcer patients.

6) Evaluation of the Effect of Antimicrobial Proteins Delivered by Amniotic Membrane on Established Infection at the Wound Site

Based on the result from experiment (1), certain titers of two important wound pathogens, S. aureus and P. aeruginosa may be inoculated at the wound sites: No antibiotics is used in this study. The infection is monitored clinically by Gram stain, selected culture of wound exudate, and histological evidence of neutrophil infiltration and cartilage necrosis. Both Gram negative P. aeruginosa and Gram positive S. aureus are tested individually and in combination in this study. Antimicrobial proteins are delivered by adenoviral-transduced amniotic membrane according to the present invention. A successful microbiological outcome may be defined as follows: partial microbiological response: 1-2 log reduction in wound colony forming units (cfu); complete microbiological response: >2 log reduction in wound cfu.

### Example 11

### Culture of Human Amniotic Epithelial Cells

Improved cell harvesting technique: Human amniotic membrane is separated from the placental tissue with blunt dissection. The isolated amniotic membrane is treated trypsin for extended period of time until small percent of amniotic epithelial cell become detached from the membrane. The trypsin treated amniotic membrane is further treated with Dispase to detach the majority of amniotic epithelial cells from the membrane. The detached amniotic epithelial cells are washed with culture medium before seeding our culture flasks. This trypsin and Dispase sequential treatment improves the quality and survival rated our isolated amniotic epithelial cells compare to published procedures (which rely on repeated and extended trypsin treatment).

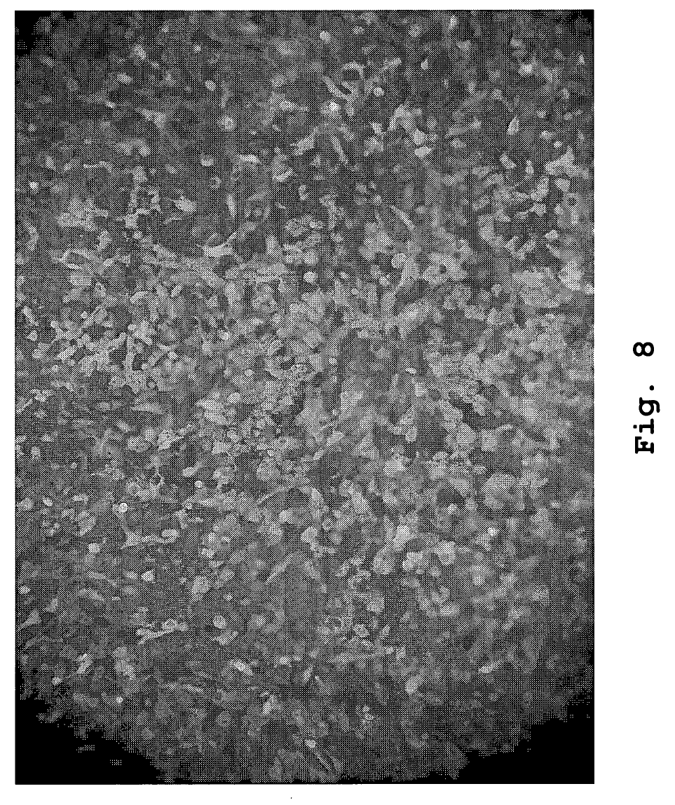

Improved culture medium for human amniotic epithelial cells: Human amniotic epithelial cells are cultured in DMEM or RPMI 1640 media supplement with 10% FBS in the most of published reports. Adding hydrocortisone and TGF-a family growth factor into the standard media can significantly increase the cell proliferation rate and help maintaining the cell morphology for amniotic epithelial cells. This improvement also enhances the transfection or retroviral transduction efficiency for the amniotic epithelial cells (FIG. 8).

Improved seeding of human amniotic epithelial cells on collagen matrix and denuded amniotic membrane: Human amniotic epithelial cells are not very effective to attach to untreated collagen matrix and denuded amniotic membrane. Coating of the surface with epithelial cell deposit basement membrane significantly increased the attachment and proliferation of amniotic epithelial cells on collagen matrix and denuded amniotic membrane. The coating of the surface is accomplished with first culturing a epithelial cell line MDCK cells onto the surface, after reaching of confluency, the MDCK cells are gently removed by treating membrane with hypotonic ammonia hydroxide in sterilized water. This gentle removal of MDCK cells exposes the basement membrane for improved attachment and function of human amniotic epithelial cells. The epithelial cells for depositing basement membrane do not limited to MDCK cells. It is preferred to use human derived epithelial cells for reducing potential immunogenicity.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and example be considered as exemplary only, with a true scope and spirit of the invention being indicated by the claims.

What is claimed is:

1. A method for delivering a molecule to a patient comprising
administering amniotic epithelial cells to the skin of the patient;
wherein said cells are capable of delivering said molecule.

2. The method of claim 1, wherein said patient is a human patient.

3. The method of claim 2, wherein said molecule is useful in achieving a desired effect.

4. The method of claim 3, wherein said cells are capable of delivering said molecule to the skin in amounts sufficient to achieve the desired effect.

5. The method of claim 4, wherein said desired effect is selected from the group consisting of a therapeutic effect, a cosmetic effect, a diagnostic effect and a prophylactic effect.

6. The method of claim 5, wherein said cells were engineered to include an exogenous polynucleotide.

7. The method of claim 3, wherein said molecule is selected from the group consisting of a growth factor, a ligand, an immunologically active molecule, an anti-microbial protein, an anti-inflammatory protein, an anti-neovascularization protein, a protease inhibitor, a hair growth promoting factor, an antiviral protein, a bioactive antibody, a bioactive single chain antibody, PDGF-beta, KGF, KGF-2, FGF-2, EGF, TGF-a, epiregulin, VEGF, NGF, GM-CSF, TGF-b, IGF-I, HGH, a bactericidal/permeability-increasing protein, a protein, a polypeptide, a peptide, a defensin, a collectin, Granulysin, Protegrin-1, SMAP-29, lactoferrin, Calgranulin C, interleukin-1 receptor antagonist, soluble TNF receptor, soluble CTLA4, interleukin-10, endostatin, angiostatin, soluble VEGF receptor, TIMPs, PAI-1, PAI-2, ecotin, wnt, sonic hedgehog, soluble herpes viral receptor Hve A, herpesvirus entry mediator C (HveC), the herpesvirus immunoglobulin-like receptor (HIgR), and soluble herpes surface protein gD.

8. The method of claim 1, wherein said amniotic epithelial cells are selected from the group consisting of human cells, animal cells, mammalian cells.

9. The method of claim 1, wherein said cells are capable of delivering said molecule in a nutrient-poor environment found on the skin.

10. The method of claim 2, wherein said cells are human amniotic epithelial cells.

11. The method of claim 1, wherein said method further comprises administering a support to said skin.

12. The method of claim 11, wherein said support is selected from the group consisting of a membrane, a matrix, a gel, a web, a net, a natural membrane, a synthetic membrane, and a material capable of performing the function of a membrane.

13. The method of claim 12, wherein said membrane is selected from the group consisting of amnion membrane, cerebral dura mater membrane, fascia lata membrane, and pericardium membrane.

14. The method of claim 6, wherein said cells were engineered using a vector.

15. The method of claim 14, wherein said vector is selected from the group consisting of a retroviral vector, an adenoviral vector, a lentiviral vector, a viral vector, an adeno-associated viral vector, a plasmid vector and a cosmid vector.

16. A composition for delivering a molecule to a patient comprising cells capable of delivering said molecule to the patient, a support capable of facilitating delivery of said molecule to the patient, wherein said cells are capable of delivering said molecule in a nutrient-poor environment found on the skin.

17. The composition of claim 16, wherein said molecule is useful in achieving a desired effect.

18. The composition of claim 17, wherein said cells are capable of delivering said molecule to the skin in amounts sufficient to achieve the desired effect.

19. The composition of claim 18, wherein said wherein said cells were engineered to include an exogenous polynucleotide.

20. The composition of claim 18, wherein said cells are human amniotic epithelial cells.
